(19) United States
(12) Patent Application Publication
(10) Pub. No.: US 2024/0294827 A1
(43) Pub. Date: Sep. 5, 2024

(54) Organic Semiconducting Compound and Organic Photoelectric Components Using the Same

(71) Applicant: Raynergy Tek Incorporation, Hsinchu (TW)

(72) Inventors: Min-Hsien Chen, Hsinchu (TW); Chuang-Yi Liao, Hsinchu (TW); Lin-Chieh Cheng, Hsinchu (TW)

(21) Appl. No.: 18/434,232

(22) Filed: Feb. 6, 2024

Related U.S. Application Data
(60) Provisional application No. 63/444,578, filed on Feb. 10, 2023.

(51) Int. Cl. C09K 11/06; H10K 50/14; H10K 85/60; H10K 102/10

(52) U.S. Cl. CPC C09K 11/06; H10K 50/14; H10K 85/615; H10K 85/655; H10K 85/656; H10K 85/6576; H10K 2102/10

(57) ABSTRACT

The present application provides an organic semiconducting compound and organic photoelectric components using the same. The organic semiconducting compound includes a novel design of chemical structure. By fabricating organic photoelectric components using the organic semiconducting compound, the heat resistance of organic photoelectric components can be enhanced and hence extending the lifetime thereof.

### FIELD OF THE INVENTION

The present invention related to a compound and photoelectric components using the same with good physical and chemical advantages, particularly good thermal stability, good for process, increasing convenience for production, and excellent thermal-resistance.

### BACKGROUND OF THE INVENTION

In recent years, in order to manufacture more versatile and lower-cost electronic components, the demand for organic semiconducting compounds has been increasing. Compared to traditional semiconductor materials, the organic semiconducting compounds have a wide light absorption range, a high light absorption coefficient, and a tunable structures. Their light absorption range, energy level, and solubility can be adjusted according to target requirements. In addition, organic compound materials have more advantages, such as low cost, flexibility, low toxicity, and large-area production in component production, making organic compound materials highly competitive in various fields. Such materials have a wide range of applications, including organic field-effect transistor (OFET), organic light-emitting diode (OLED), organic photodetector (OPD), organic photovoltaic cells (OPV), sensors, storage components, and various components in logic circuits. In general, organic semiconductor materials are usually present in the form of thin films with a thickness of about 50 nm to 1 μm in the above applications of the organic semiconductor components.

Where, the active layer material in organic photoelectric components directly influences the device performance and therefore plays an important role. The material can be divided into two parts, donor and acceptor. Common donor materials include organic polymers, oligomers, or confined molecular units. Currently, the development of D-A (Donor-Acceptor) type conjugated polymers is the mainstream. The push-pull electron effect formed by the interaction between electron-rich units and electron-deficient units in polymers can be used to adjust the energy level and energy gap of the polymers. The matched acceptor material is usually a fullerene derivative with high conductivity, and its light absorption range is about 400-600 nm. Besides, graphene, metal oxides or quantum dots can be concluded in the matched acceptor material as well.

However, fullerenes and their derivatives are difficult to be synthesized and purified, and their structure is not easy to be adjusted. The ranges of their absorption wavelength and energy-level are also limited. The absorption is very weak when it is greater than 700 nm, which limits the matching of donor and acceptor materials. With the development of the market, the demand for materials in the near-infrared (NIR) region is gradually increasing. Even if the absorption range of the conjugated polymer donor material can be adjusted to the near-infrared region, it is still limited by the fact that the fullerene acceptor may not be matched with the donor well. Therefore, the development of non-fullerene acceptor compounds to replace traditional fullerene acceptor compounds is especially important in the breakthrough of active layer materials.

Despite this, the early development of non-fullerene acceptor compounds was quite difficult because it was not easy to control the morphology, and therefore their power conversion efficiency was low. Fortunately, the number of studies on non-fullerene acceptors has boomed since 2015, making their electrical properties significantly improved and becoming a competitive choice. This change is mainly attributed to advances in synthesis methods and improvements in material design strategies. The extensive range of donor materials previously developed for fullerene acceptors has also indirectly contributed to the development of non-fullerene acceptor compounds.

The current development of non-fullerene acceptor compound materials mainly uses an electron-rich core with electron-deficient units on both sides to form a molecule with an A-D-A structure. D is usually a molecule composed of a benzene ring and thiophene, and A is usually a cyano-indanone (IC) derivative. Another type of structure is using the A′-D-A-D-A′ pattern as the electron-deficient unit at the center. Molecules containing sulfur atoms, nitrogen atoms, or selenium atoms are often used to enhance its performance.

In order for organic photoelectric components or devices to be commercialized, their materials should be conducive to production, and these materials must have considerable thermal stability to achieve the required product lifetime. Therefore, how to improve the thermal stability of materials has become a current issue. As for the existing acceptor materials, they can be roughly divided into two types: small molecules and polymers. When the components are heat-treated, the small molecule type of acceptors easily form molecular crystals due to their physical properties, causing device defects. When manufacturing polymer-type acceptors, due to the preparation method, it is difficult to control the polymerization results in each batch of production and the product reproducibility is poor.

In addition, in response to the requirements of environmental protection regulations in various countries and the requirements for good processing operability, the material manufacturing process must be conducive to wet process operations. Therefore, developing an organic semiconducting compound that has better thermal stability and can use non-halogen solvents is a problem that those skilled in the art want to solve.

### SUMMARY OF THE INVENTION

An objective of the present application is to provide an organic semiconducting compound, particularly an n-type organic semiconducting compound, capable of overcoming the drawbacks of the conventional organic semiconducting compound and providing one or multiple advantages as described above, in particular, thermal stability. It is synthesized by a method suitable for mass production and shows good processability, which is conducive to large-scale manufacturing using solution processing.

Another objective of the present application is to provide a novel organic photoelectric component formed by the organic semiconducting compound according to the present application and exhibiting higher heat resistance than the organic photoelectric component according to the prior art.

To achieve the above objective, the present application provides an organic semiconducting compound, which comprises:

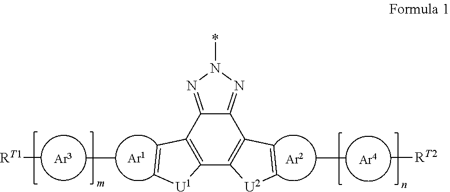

and a connecting unit, which is connected to two or more units of Formula 1;
wherein
U¹, U² are NR¹, C═O, O, S, Se, or CR¹R²;

R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group;

Ar¹, Ar² are substituted or unsubstituted arylene or heteroarylene group with 5 to 20 ring atoms, which is a monocyclic, polycyclic, or fused ring;

Ar³, Ar⁴ are independently selected from at least one of —CY¹═CY²—, —C≡C—, substituted monocyclic, polycyclic, or fused ring arylene with 5 to 20 ring atoms, or substituted monocyclic, polycyclic, or fused ring heteroarylene with 5 to 20 ring atoms;
Y¹, Y² are H, F, Cl, or CN;
R^{T1}, R^{T2} are electron-withdrawing group;
* is the bonding position to the connecting unit; and
m, n are integers between 0 and 5.

To achieve another objective of the present application as described above, the present application further provides an organic semiconducting component, which comprises a substrate, an electrode module, and an active layer. The electrode module is disposed on the substrate and includes a first electrode and a second electrode. The active layer is disposed between the first electrode and the second electrode. The material of the active layer comprises at least one organic semiconducting compound as described above. Besides, one or more of the first electrode and the second electrode is transparent or translucent.

### BRIEF DESCRIPTION OF DRAWINGS

FIG. 1 shows a structural schematic diagram of the organic photoelectric component according to a first embodiment of the present application;

FIG. 2 shows a structural schematic diagram of the organic photoelectric component according to a second embodiment of the present application;

FIG. 3 shows a structural schematic diagram of the organic photoelectric component according to a third embodiment of the present application;

FIG. 4 shows a structural schematic diagram of the organic photoelectric component according to a fourth embodiment of the present application;

FIG. 5 shows a structural schematic diagram of the organic photoelectric component according to a fifth embodiment of the present application;

FIG. 6 shows a structural schematic diagram of the organic photoelectric component according to a sixth embodiment of the present application;

FIG. 7A shows the experimental results of absorption spectrum for the organic semiconducting compound N1 according to the present application;

FIG. 7B shows the experimental results of absorption spectrum for the organic semiconducting compound N2 according to the present application;

FIG. 7C shows the experimental results of absorption spectrum for the organic semiconducting compound N3 according to the present application;

FIG. 7D shows the experimental results of absorption spectrum for the comparative compound 1; and

FIG. 7E shows the experimental results of absorption spectrum for the comparative compound 2.

### DETAILED DESCRIPTION OF THE INVENTION

According to the prior art, the organic semiconductor materials that use polymer types or small molecule types with donor-acceptor structures are affected by the tendency of small molecule types to form ordered stacks and the lack of good reproducibility of polymer types, imposing negative influence on the heat resistance of the organic photoelectronic components formed by using the materials.

The advantage of the present application is that the organic semiconducting compound according to the present application utilizes the modification characteristic of the No. 2 nitrogen atom of the triazole functional group in its unit structure, and connects them with each other through a connecting unit to increase the molecular weight, thereby increasing the thermal stability of the organic semiconducting compounds. In addition to being easy to synthesize, the fabrication process exhibits good processability and good solubility in solvents, which is also conducive to large-scale manufacturing using solution processing methods.

First, the organic semiconducting compound according to the present application comprises:

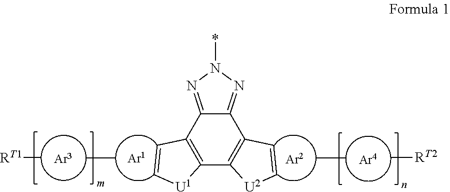

and a connecting unit, which is connected to two or more units of Formula 1;
wherein
U¹, U² are NR¹, C═O, O, S, Se, or CR¹R²;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group;
Ar¹, Ar² are substituted or unsubstituted arylene or heteroarylene group with 5 to 20 ring atoms, which is a monocyclic, polycyclic, or fused ring;
Ar³, Art are independently selected from at least one of —CY¹═CY²—, —C≡C— substituted monocyclic, polycyclic, or fused ring arylene with 5 to 20 ring atoms, or substituted monocyclic, polycyclic, or fused ring heteroarylene with 5 to 20 ring atoms;
Y¹, Y² are H, F, Cl, or CN;
R^{T1}, R^{T2} are electron-withdrawing group;
* is the bonding position to the connecting unit; and
m, n are integers between 0 and 5.

Preferably, the connecting unit of the organic semiconducting compound according to the present embodiment is selected from at least one of:

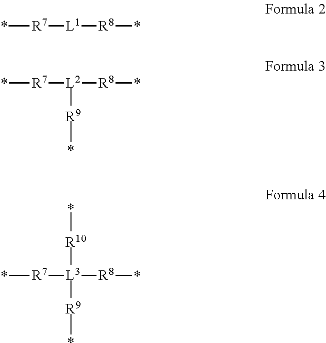

wherein
R⁷⁻¹⁰ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜ C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, and C3˜C30 alkyl group substituted by ketone group;
L¹ is selected from at least one of CR^{a}R^{b}, NR^{a}, O, SiR^{a}R^{b}, SO₂, (O═C)R^{a}R^{b} and A¹;
L² is selected from at least one of CR^{c}, N, SiR^{c}, P, P(O)₃, O═P(O)₃ and A²;
L³ is C, Si, A³;
* are bonding positions;
A¹-A³ are non-halogen-substituted aromatic ring groups with 5 to 20 ring atoms, monohalogen-substituted, or polyhalogen-substituted; and
R^{a}, R^{b}, R^{c} are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 5 to 20 ring atoms.

More preferably, the connecting unit as described above is selected from at least one of:

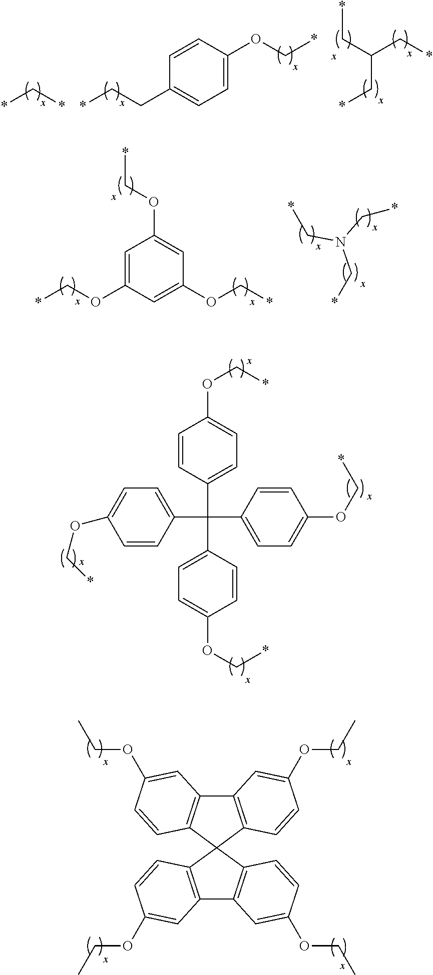

and x is an integer between 1 and 30, and * are bonding positions.
Preferably, Ar¹ of the organic semiconducting compound according to the present embodiment is selected from at least one of:

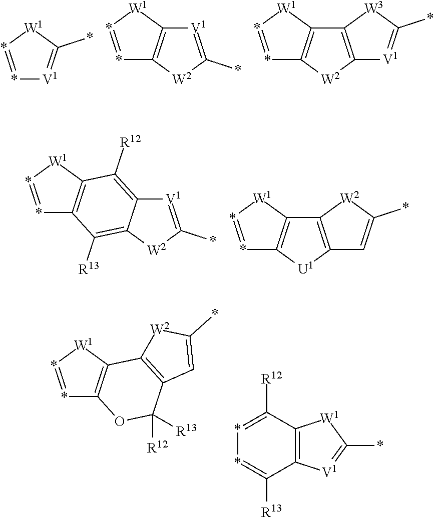

wherein
W¹⁻³ are O, S, or Se;
U¹ is NR¹, C═O, O, S, Se, or CR¹R²;
V¹ is N, CR¹;
* are bonding positions;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
R¹²⁻¹³ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

More preferably, Ar¹ is selected from at least one of:

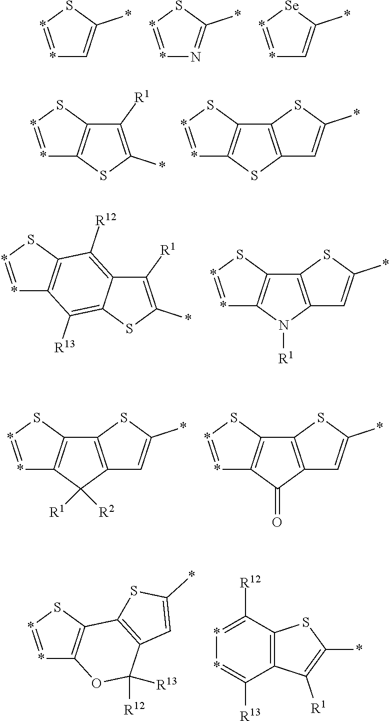

wherein
* are bonding positions;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
R¹²⁻¹³ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

Preferably, Ar² of the organic semiconducting compound according to the present embodiment is selected from at least one of:

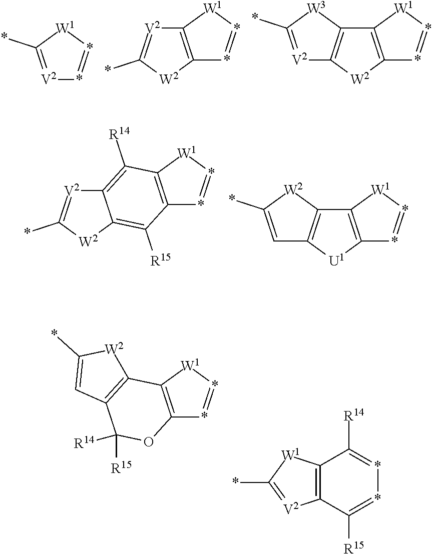

wherein
W¹⁻³ are O, S, or Se;
U¹ is NR¹, C═O, O, S, Se, or CR¹R²;
V² is N, CR¹;
* are bonding positions;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
R¹⁴⁻¹⁵ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

More preferably, Ar² is selected from at least one of:

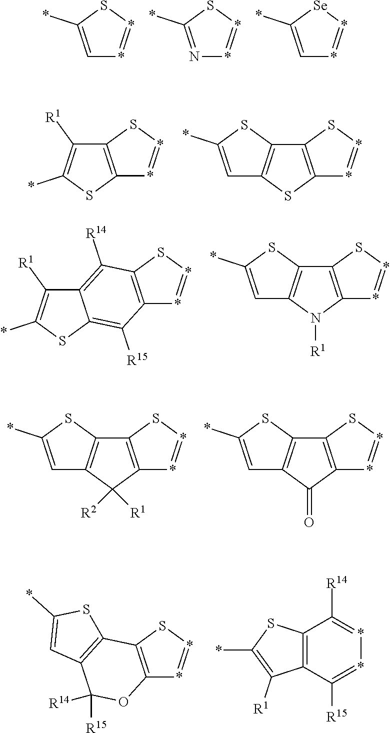

wherein
* are bonding positions;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
R¹⁴⁻¹⁵ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

Preferably, Ar³-4 of the organic semiconducting compound according to the present embodiment are selected from at least one of:

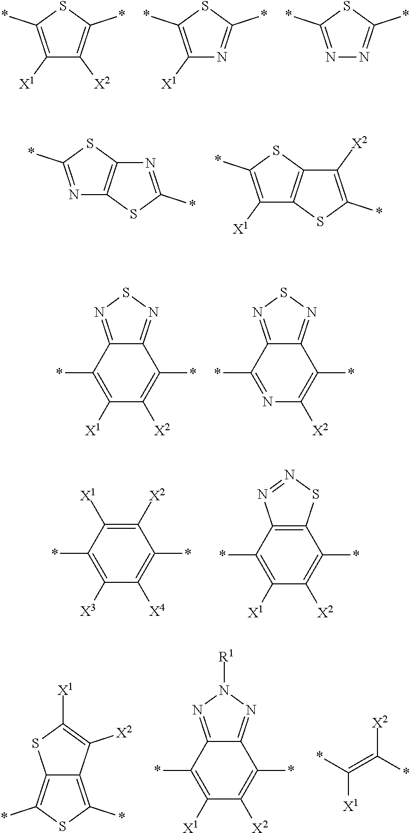

wherein
* are bonding positions;
R¹ is independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
X¹, X² are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, and C3˜C30 alkyl group substituted by ketone group.

More preferably, Ar³⁻⁴ are selected from at least one of:

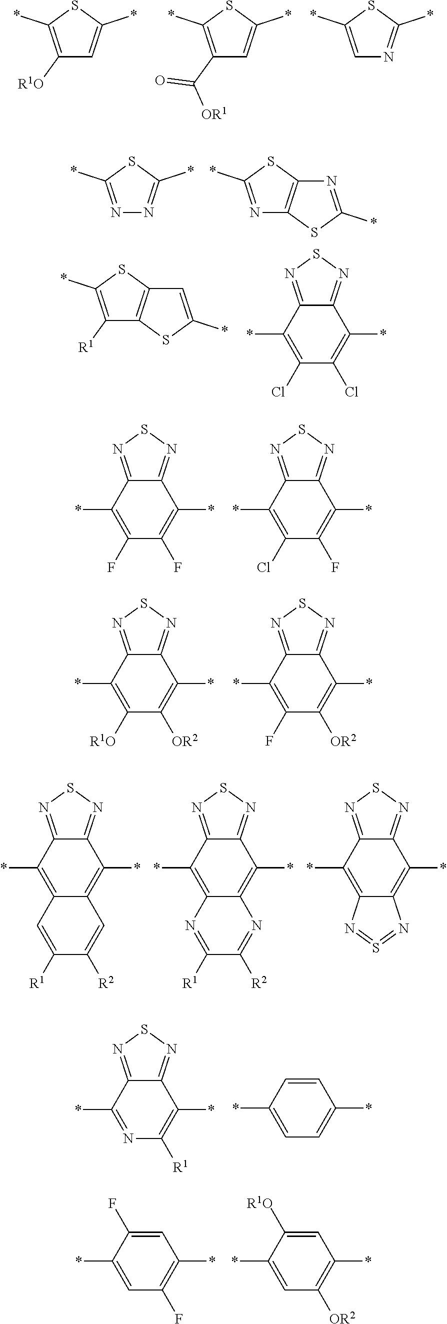

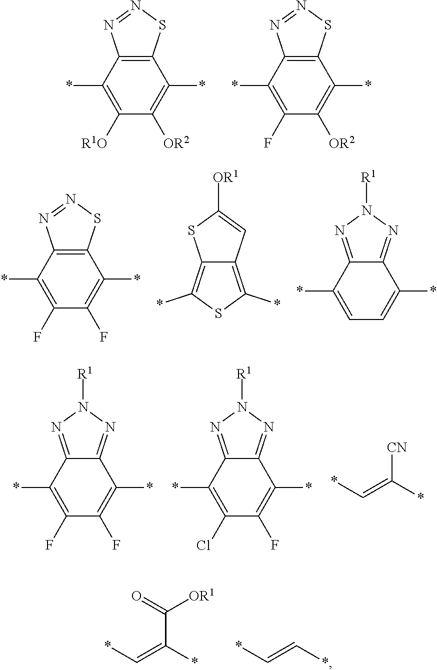

Wherein
* are bonding positions; and
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group.

Preferably, R^{T1} and R^{T2} of the organic semiconducting compound according to the present embodiment are selected from at least one of:

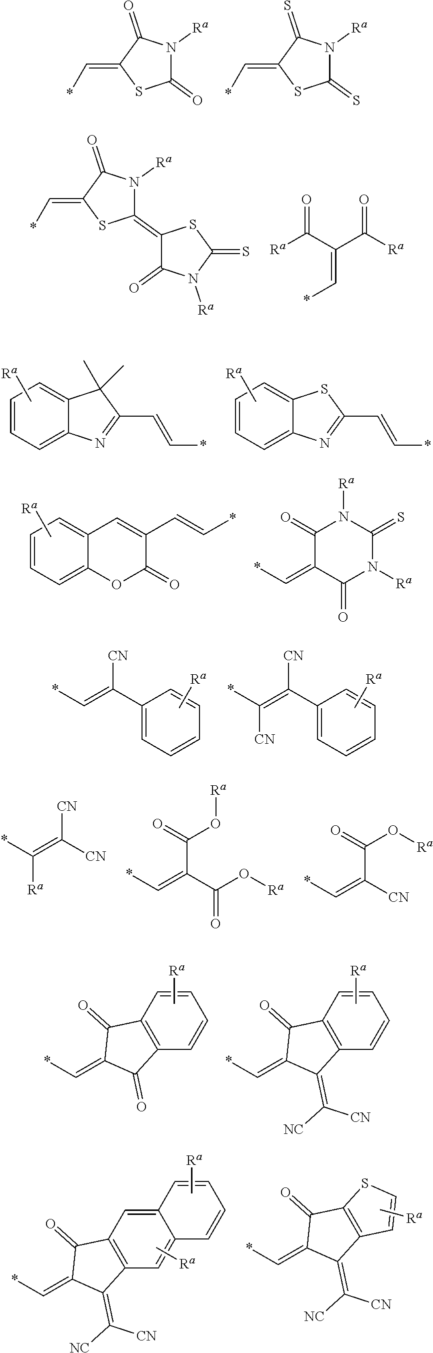

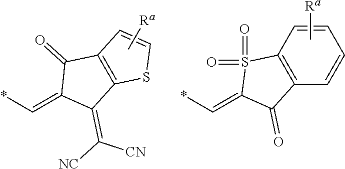

Wherein * are bonding positions; and

R^{a} is independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 5 to 20 ring atoms.

More preferably, RTI and R^{T2} are selected from at least one of:

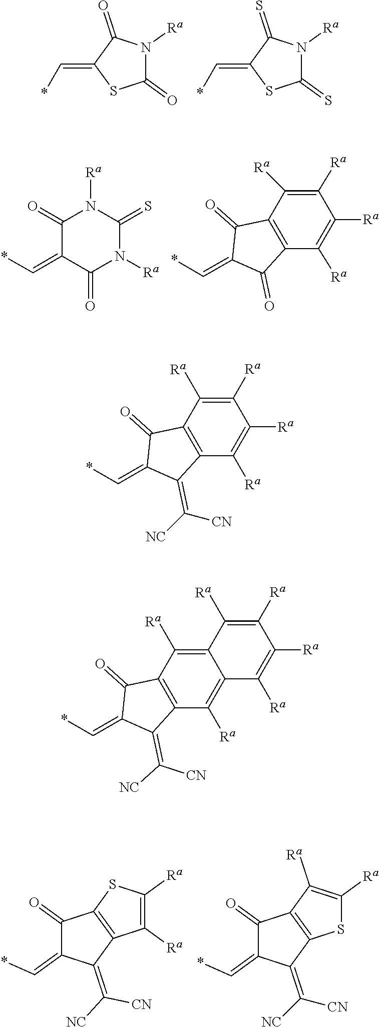

Wherein * are bonding positions; and
R^{a} is independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 5 to 20 ring atoms.

In the following, examples of methods for preparing the organic semiconducting compound according to the present application will be illustrated.

### Preparation of the Compound N1:

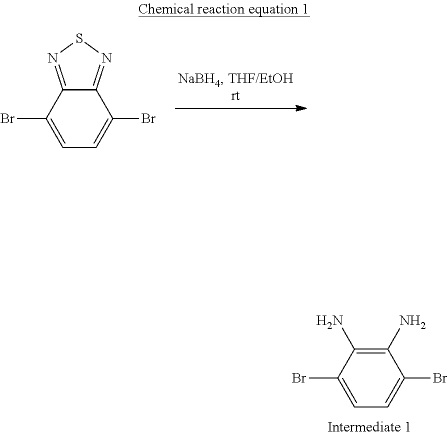 [SMILES: Brc1ccc(Br)c2nsnc12 ; Nc1c(Br)ccc(Br)c1N]

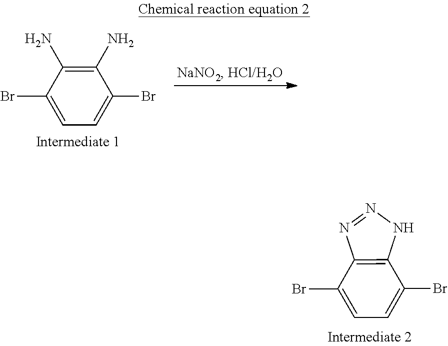 [SMILES: Nc1c(Br)ccc(Br)c1N ; Brc1ccc(Br)c2[nH]nnc12]

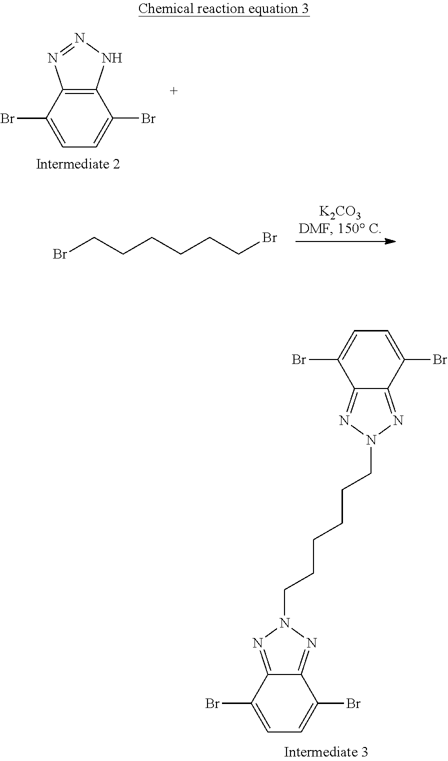 [SMILES: Brc1ccc(Br)c2[nH]nnc12 ; BrCCCCCCBr ; Brc1ccc(Br)c2nn(CCCCCCn3nc4c(Br)ccc(Br)c4n3)nc12]

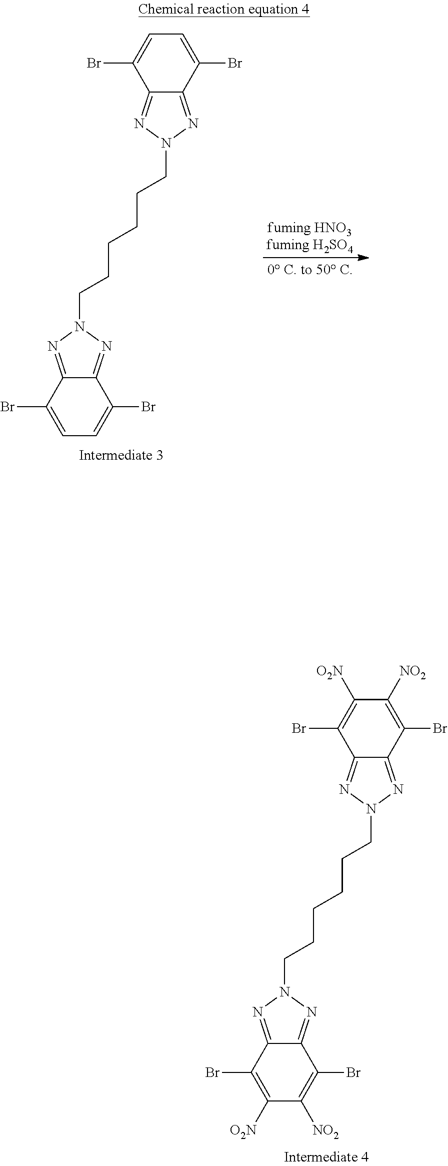 [SMILES: Brc1ccc(Br)c2nn(CCCCCCn3nc4c(Br)ccc(Br)c4n3)nc12 ; O=[N+]([O-])c1c([N+](=O)[O-])c(Br)c2nn(CCCCCCn3nc4c(Br)c([N+](=O)[O-])c([N+](=O)[O-])c(Br)c4n3)nc2c1Br]

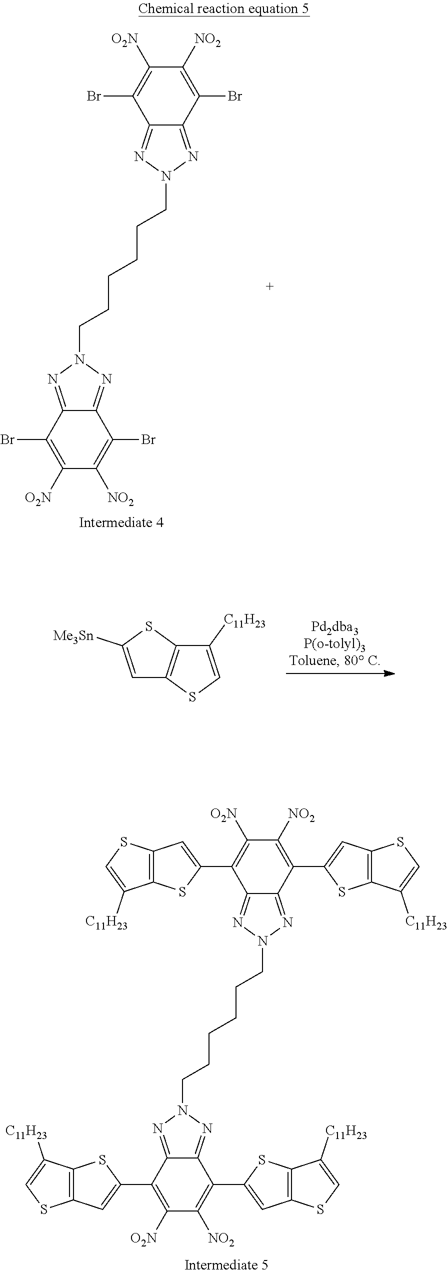

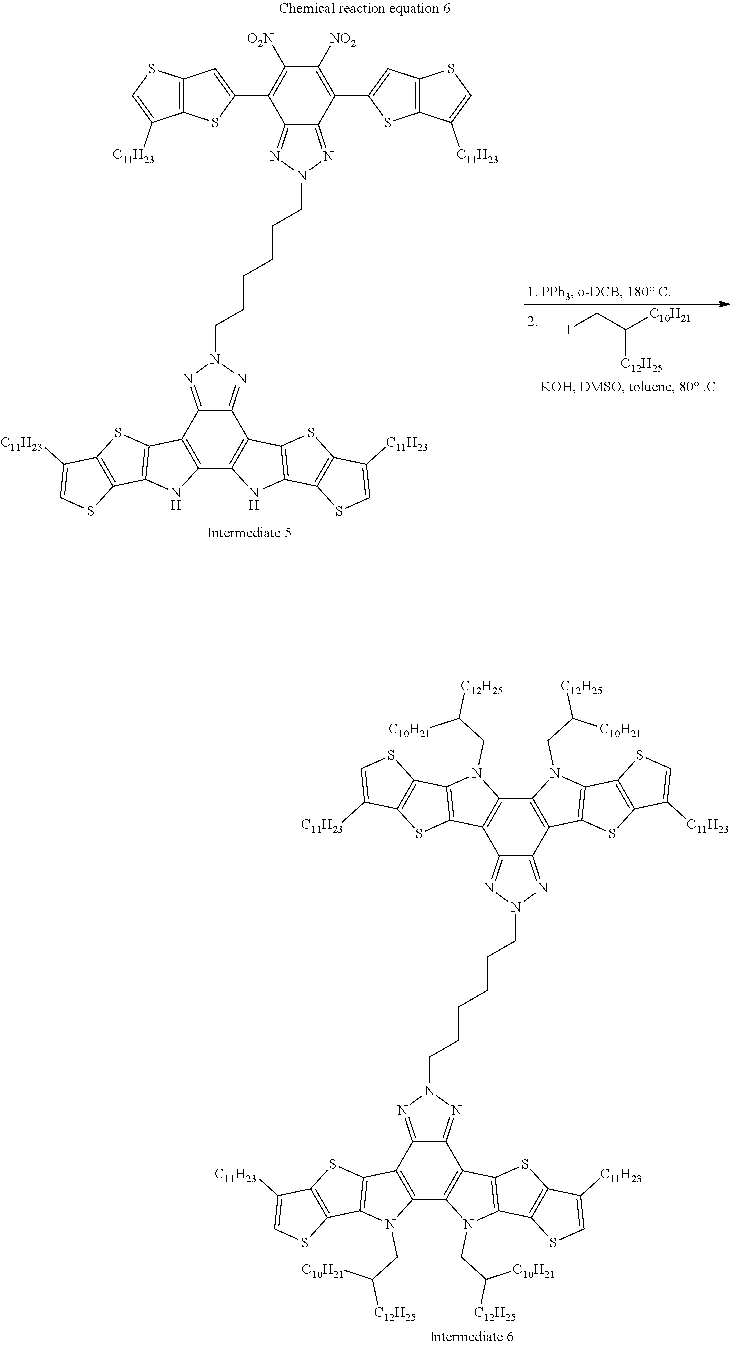

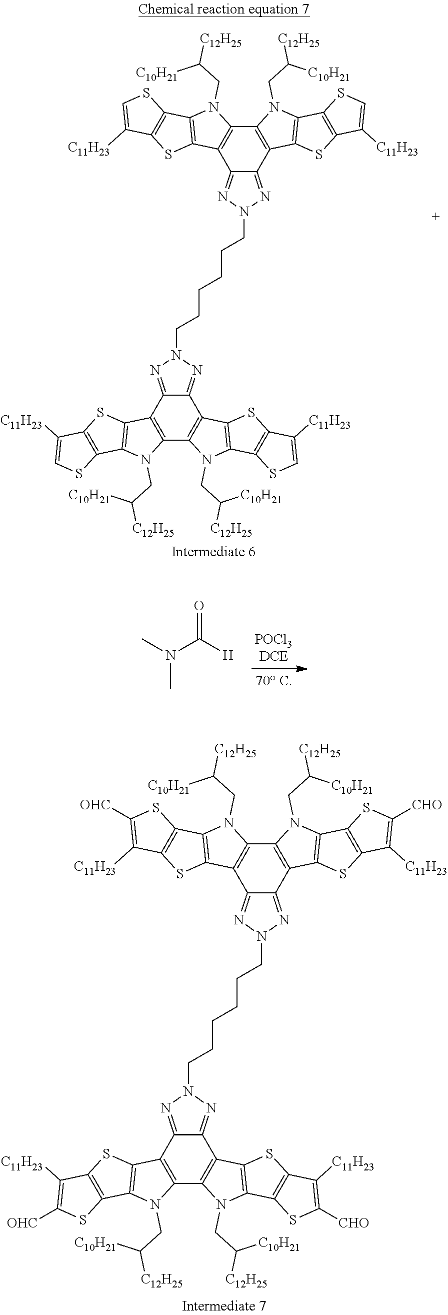

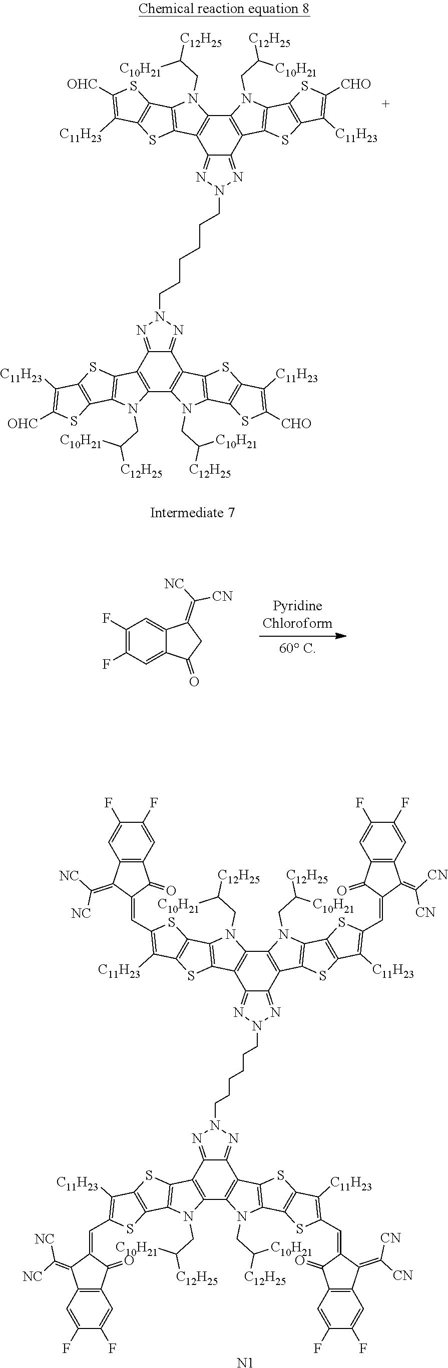

First, the chemical reaction equation 1 is described as follows:

In a round-bottomed flask, 4,7-dibromo-2,1,3-benzothiadiazole (1.51 g, 5.14 mmol) is dissolved in tetrahydrofuran (38 ml) and ethanol (23 ml) at room temperature and sodium borohydride (3.89 g, 102.74 mmol) is added in portions. After one hour, water (75 ml) is added to quench the reaction, and the mixture is extracted with ethyl acetate (3×100 ml). The combined organic layer is dried by anhydrous magnesium sulfate, filtered, and the solvent is removed under vacuum to obtain Intermediate 1 (1.09 g, 80%). 1H NMR (600 MHz, CDCl₃): 6.85 (2H, s), 3.90 (4H, s).

The chemical reaction equation 2 is described as follows:

Add 37% HCl (20 ml) and water (35 ml) into the round-bottomed flask containing Intermediate 1 (1.00 g, 3.76 mmol). Dissolve sodium nitrite (1.17 g, 16.92 mmol) in water (35 ml) and slowly add it to the above-mentioned round-bottom bottle at room temperature. When Intermediate 1 is completely consumed, add water (500 ml) to dilute the mixture and precipitate the product. Filter the product with suction funnel and rinse the solid with water. Remove the solvent from the collected solid under vacuum to obtain Intermediate 2 (1.03 g, 99%). 1H NMR (600 MHz, CDCl₃): 7.55 (2H, s).

The chemical reaction equation 3 is described as follows:

Add Intermediate 2 (1.50 g, 5.42 mmol) and potassium carbonate (5.98 g, 43.33 mmol) into a double-necked flask, evacuate the flask and fill it with argon. After adding dimethylformamide (23 ml) and 1,6-dibromohexane (0.66 g, 2.71 mmol) sequentially under argon, the flask is moved to a 150-degree oil bath for reaction. When the reaction is completed, cool down and extract with dichloromethane (3×100 ml) and water (200 ml). The collected organic layer is dried with MgSO₄ then filtered, and the solvent is removed under vacuum. The crude product is purified by silica gel column chromatography (dichloromethane) to obtain the product Intermediate 3 (0.50 g, 29%). ¹H NMR (500 MHZ, CDCl₃): 7.42 (4H, s), 4.76 (4H, t, J=6.0 Hz), 2.14 (4H, t, J=7.0 Hz), 1.45-1.42 (4H, m).

The chemical reaction equation 4 is described as follows:

Under an ice bath, slowly add fuming nitric acid (5.6 ml) to the round-bottomed flask containing oleum (0.8 ml). Add Intermediate 3 (0.80 g, 1.26 mmol) into another round-bottomed bottle and slowly add the mixed acid into the bottle under an ice bath. After the addition is complete, move the reaction flask to an oil bath at 50 degrees. After the reaction is completed, the mixture is slowly dripped into ice. The precipitated solid is collected by suction funnel filtration and rinsed with water. The solid is collected and dried under vacuum to obtain Intermediate 4 (0.76 g, 74%). 1H NMR (500 MHz, C₂D₂Cl₄): 4.88 (4H, t, J=7.0 Hz), 2.22-2.20 (4H, m), 1.48-1.47 (4H, m).

The chemical reaction equation 5 is described as follows:

Intermediate 4 (0.70 g, 0.86 mmol), tris(dibenzylideneacetone)dipalladium (31 mg, 0.034 mmol), and tris(o-tolyl)phosphine (41 mg, 0.14 mmol) are added into a double-necked bottle which is degas with vacuum and backfill with argon three times. Toluene (10.5 ml) and 6-undecylthieno[3,2-b]thiophen-2-yl)trimethylstannane (1.96 g, 4.29 mmol) are added sequentially under argon. The reaction is moved to an 80° C. oil bath for reaction under argon protection. When the reaction is completed, cool down the reaction, filter the mixture through Celite® and silica gel, and rinse with dichloromethane (3×100 ml). The solvent is removed from the collected filtrate and methanol (300 ml) is added to the obtained crude product to precipitate the product. The precipitated solid is collected by suction funnel filtration and rinsed with methanol (2×100 ml). The collected solid is dried under vacuum to obtain Intermediate 5 (1.41 g, 98%). 1H NMR (600 MHZ, CDCl₃): 7.73 (4H, s), 7.12 (4H, s), 4.83 (4H, t, J=7.2 Hz), 2.72 (8H, t, J-7.8 Hz), 2.17 (4H, t, 6.6 Hz), 1.77-1.72 (12H, m), 1.38-1.20 (60H, m), 0.89-0.88 (16H, m).

The chemical reaction equation 6 is described as follows:

Put Intermediate 5 (1.36 g, 0.81 mmol), triphenylphosphine (2.13 g, 8.14 mmol) and o-dichlorobenzene (40 ml) into a double-necked flask and heat to 180 degrees, when the starting materials are completely consumed, the solvent is distilled off, and methanol (300 ml) is added to the crude product to precipitate the product. The precipitate is collected by suction funnel filtration and rinsed with methanol (3×100 ml). The solid is collected and dried under vacuum and then placed in a double-necked flask and add potassium hydroxide (0.43 g, 7.55 mmol). Evacuate and backfill with argon three times. Toluene (10 ml) and dimethylsulfoxide (10 ml) are added under argon and stirred at room temperature for 30 minutes. Then 1-iodo-2-decyl-tetradecane (5.85 g, 12.58 mmol) is added and the mixture is heated to 80° ° C. When the reaction is completed, the mixture is extracted with n-heptane (3×100 ml) and water. The collected organic layer is dried by anhydrous magnesium sulfate and filtered. The crude product is purified by silica gel column chromatography (n-heptane:ethyl acetate=8:2) and get Intermediate 6 (0.55 g, 30%) can be obtained. 1H NMR (600 MHz, CDCl₃): 6.96 (4H, s), 4.84 (4H, t, J=7.2 Hz), 4.56 (8H, d, J=7.2 Hz), 2.80 (8H, t, J=7.8 Hz), 2.34-2.27 (4H, m), 2.02-1.98 (4H, m), 1.85 (8H, quintet, J=11.4 Hz, J=7.8 Hz), 1.67-1.63 (4H, m), 1.44 (8H, quintet, J=10.8 Hz, 7.2 Hz), 1.39-0.86 (252H, m).

The chemical reaction equation 7 is described as follows:

Phosphorus oxychloride (0.74 ml) is slowly added to the round-bottomed flask containing dimethylformamide (6.2 ml) in an ice bath. After the addition is completed, return to the room temperature and stir for 30 minutes. Add the prepared Vilsmeier reagent into a reaction flask containing Intermediate 6 (0.55 g, 0.19 mmol) and dichloroethane (15 ml), and heat to 70 degrees. At the end of the reaction, water is added to quench the reaction. The mixture is extracted with n-heptane (3×100 ml) and water. The collected organic layer is dried by anhydrous magnesium sulfate and filtered. The crude product is purified by silica gel column chromatography (n-heptane:ethyl acetate=8:2) to obtain Intermediate 7 (0.46 g, 80%). 1H NMR (600 MHz, CDCl₃): 10.12 (4H, s), 4.85 (4H, t, J=7.8 Hz), 4.59 (8H, d, J=7.2 Hz), 3.17 (8H, t, J-7.8 Hz), 2.35-2.28 (4H, m), 1.98-1.88 (12H, m), 1.67-1.64 (4H, m), 1.46 (8H, quintet, J=10.8 Hz, J=7.2 Hz), 1.38-0.85 (252H, m).

The chemical reaction equation 8 is described as follows:

Intermediate 7 (0.16 g, 0.052 mmol) and 5,6-difluoro-3-(dicyanomethylene)inden-1-one (0.12 g, 0.52 mmol) are added into a double-necked flask. Evacuate and backfill with argon three times. Add chloroform (8 ml) and pyridine (0.2 ml) sequentially into the bottle, and the mixture is reacted at 60° C. When the starting material is consumed, the reaction is cooled and methanol (200 ml) is added to precipitate the solid. Collect the solid by suction funnel filtration and rinse with methanol (3×100 ml). The collected solid is purified by column chromatography (the eluent is chloroform) to obtain compound N1 (0.10 g, 52%). 1H NMR (600 MHz, CDCl₃): 9.07 (4H, s), 8.52 (4H, q, J=9.0 Hz, J=6.0 Hz), 7.69 (4H, t, J=7.2 Hz), 4.83 (4H, t, J=7.2 Hz), 4.76 (8H, d, J=7.8 Hz), 3.10 (8H, t, J=7.8 Hz), 2.36-2.34 (4H, m), 2.07-2.06 (4H, m), 1.81-1.79 (8H, m), 1.64-1.62 (4H, m), 1.45-1.43 (12H, m), 1.25-0.79 (248H, m).

### Preparation of the Compound N2:

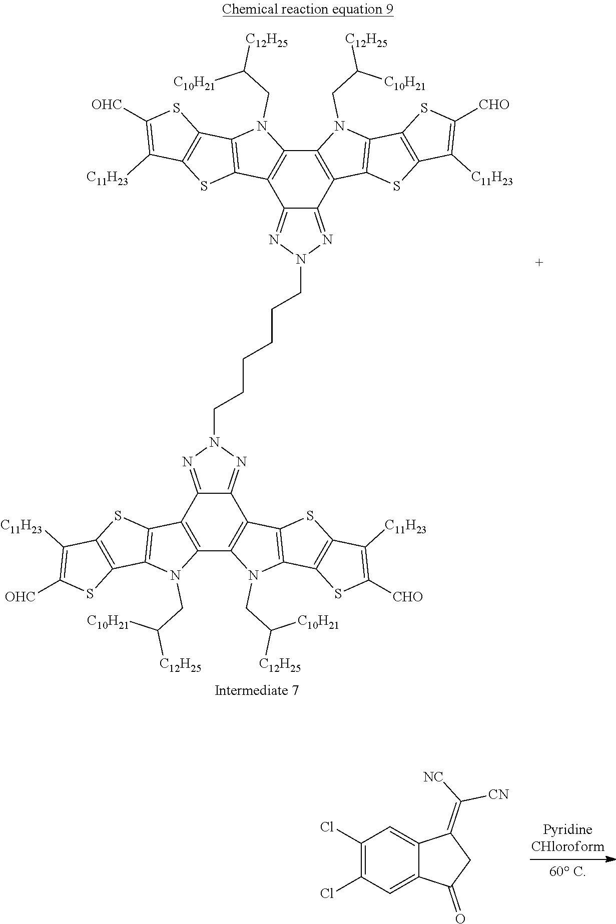

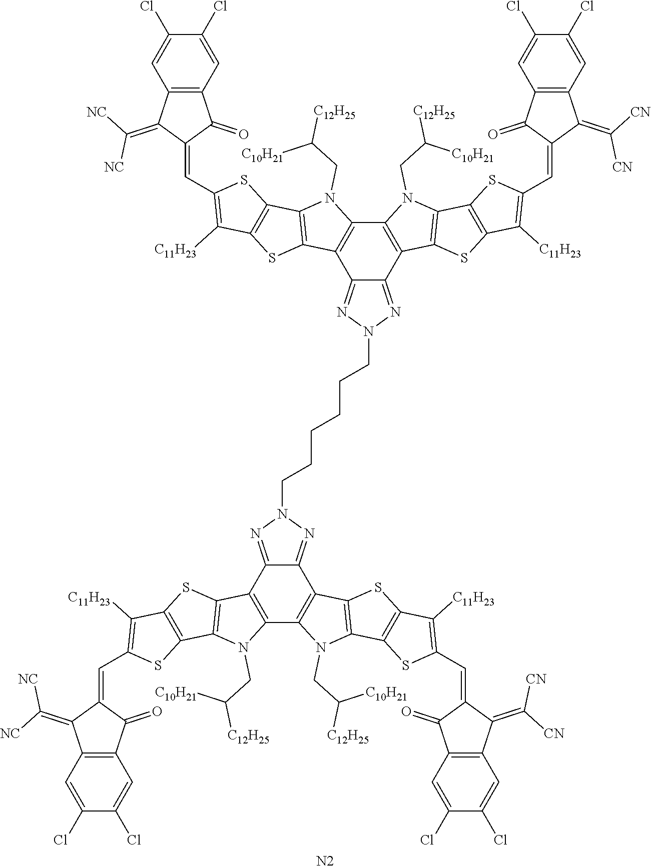

Intermediate 7 (0.16 g, 0.052 mmol) and 5,6-dichloro-3-(dicyanomethylene)inden-1-one (0.14 g, 0.52 mmol) are added into a double-necked flask. Evacuate and backfill with argon three times. Add chloroform (8 ml) and pyridine (0.2 ml) sequentially into the bottle, and the mixture is reacted at 60° C. When the starting material is consumed, the reaction is cooled and methanol (200 ml) is added to precipitate the solid. Collect the solid by suction filtration and rinse with methanol (3×100 ml). The collected solid is purified by column chromatography (the eluent is chloroform) to obtain compound N2 (0.15 g, 68%). 1H NMR (500 MHz, CDCl₃): 9.02 (4H, s), 8.72 (4H, s), 7.95 (4H, s), 4.81-4.79 (16H, m), 3.01 (8H, t, J=7.0 Hz), 2.40-2.30 (4H, m), 2.15-2.10 (4H, m), 1.77-1.74 (12H, m), 1.60-1.58 (4H, m), 1.40-1.37 (16H, m), 1.25-0.83 (236H, m).

### Preparation of the Compound N3:

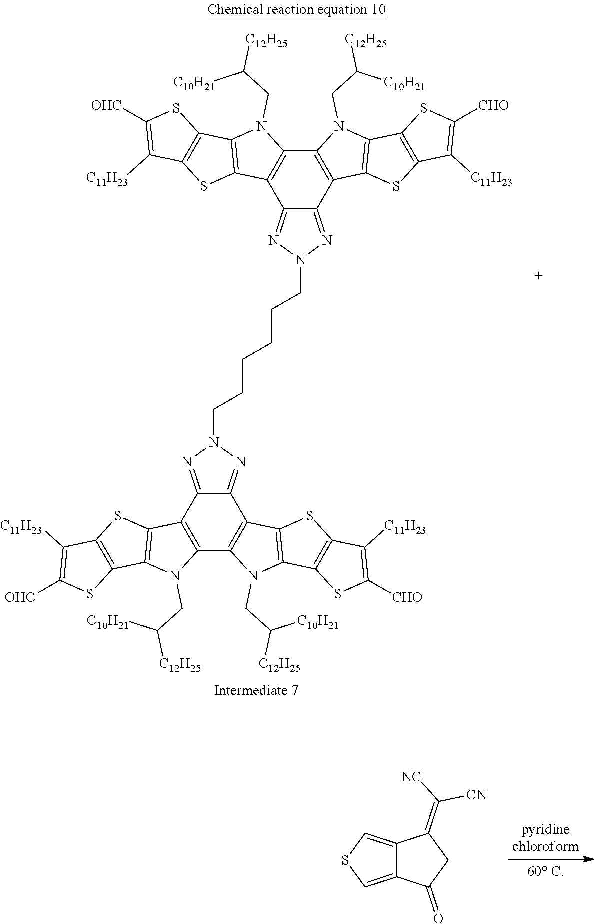

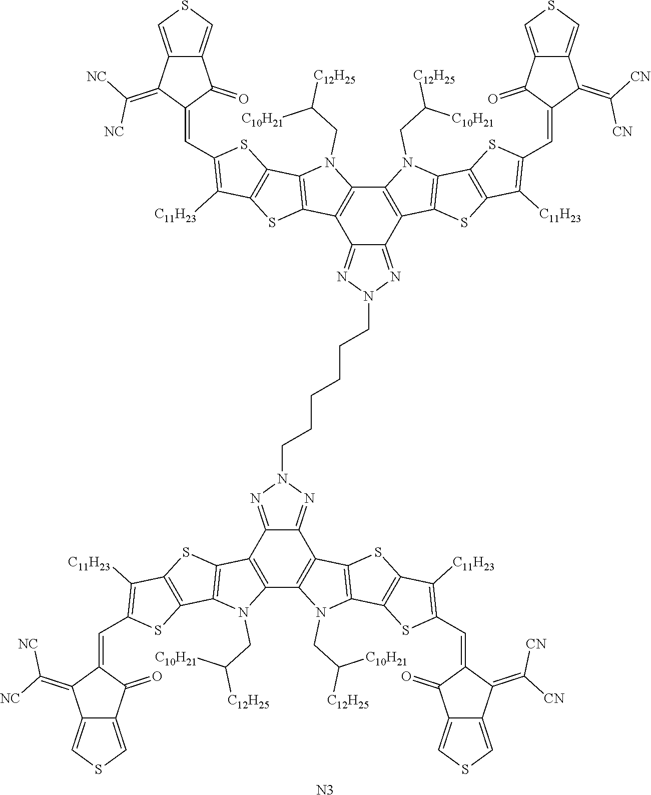

Intermediate 7 (0.16 g, 0.052 mmol), 2-(6-oxo-5,6-dihydro-4H-cyclopentyl[c]thiophene-4-ylidene)malononitrile (0.11 g, 0.53 mmol) are added into a double-neck flask. Evacuate and backfill with argon three times. Add chloroform (8 ml) and pyridine (0.2 ml) sequentially into the bottle, and the mixture is reacted at 60° C. When the starting material is consumed, the reaction is cooled and methanol (200 ml) is added to precipitate the solid. Collect the solid by suction filtration and rinse with methanol (3×100 ml). The collected solid is purified by column chromatography (the eluent is chloroform) to obtain compound N3 (0.07 g, 33%). 1H NMR (600 MHz, CDCl₃): 8.98 (4H, s), 8.31 (4H, d, J=2.4 Hz), 7.93 (4H, d, J=2.4 Hz), 4.82 (4H, t, J=7.2 Hz), 4.73 (8H, d, J=7.8 Hz), 3.06 (8H, t, J=7.2 Hz), 2.36-2.35 (4H, m), 2.10-2.09 (4H, m), 1.61-1.60 (4H, m), 1.43-1.40 (12H, m), 1.25-0.82 (256H, m).

The embodiments of the organic semiconducting compound according to the present application are shown in Table 1.

TABLE 1
Embodiments of the organic semiconducting compound according to the
present application

| | |
|---|---|
| N1: | 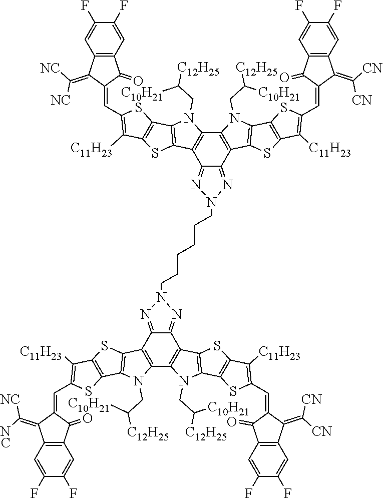 |
| N2: | 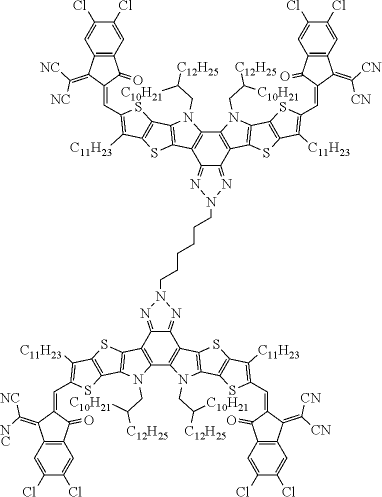 |
| N3: | 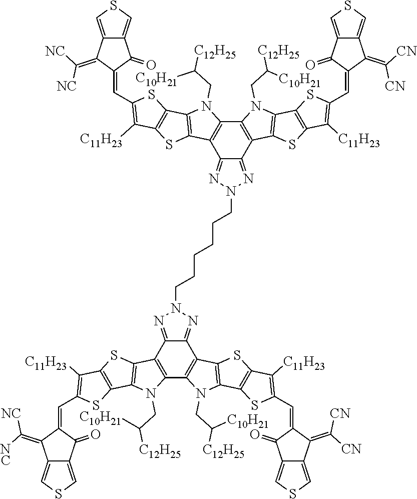 |
| N4: | 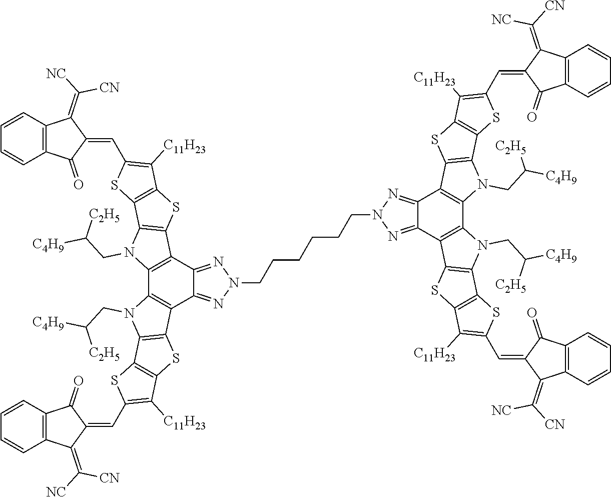 |
| N5: | 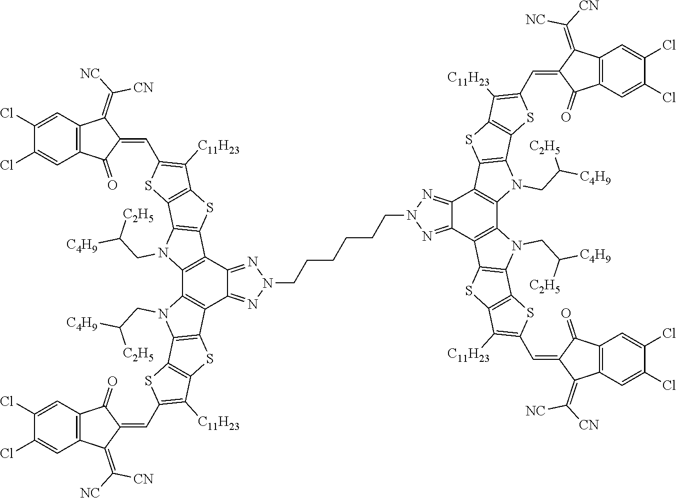 |
| N6: | 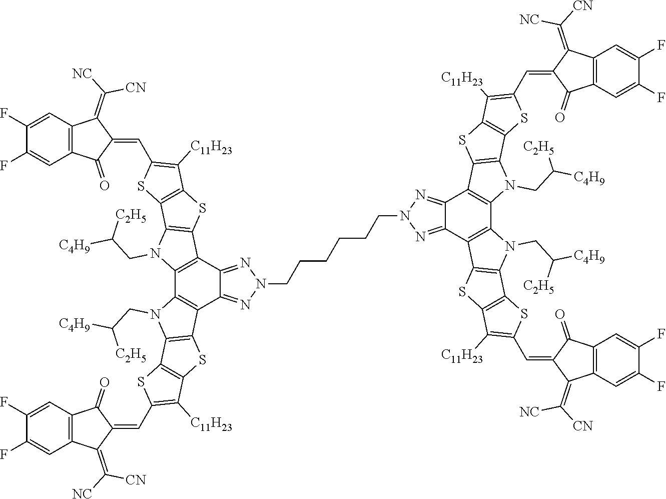 |
| N7: | 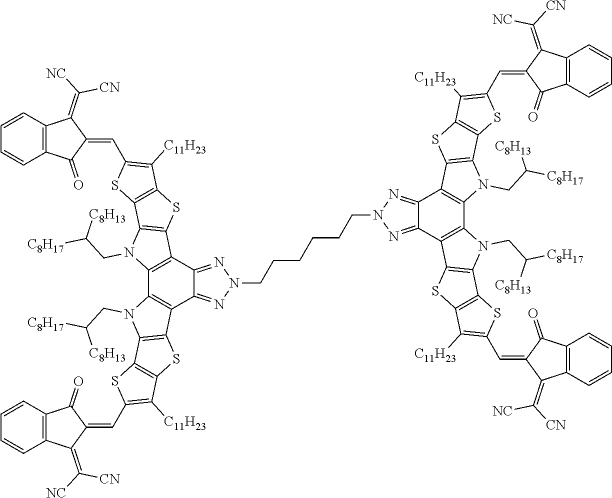 |
| N8: | 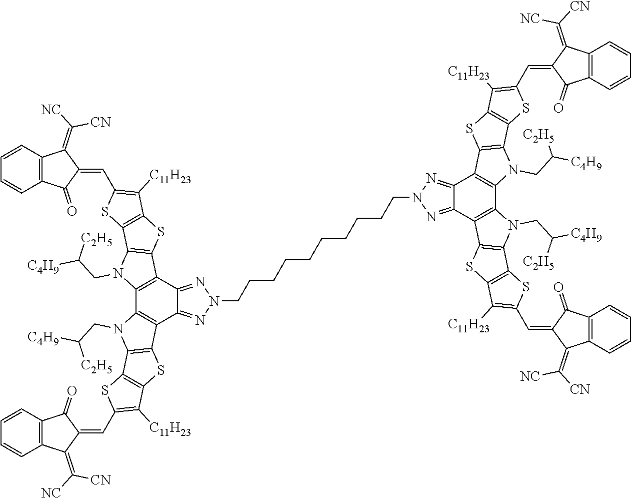 |
| N9: | 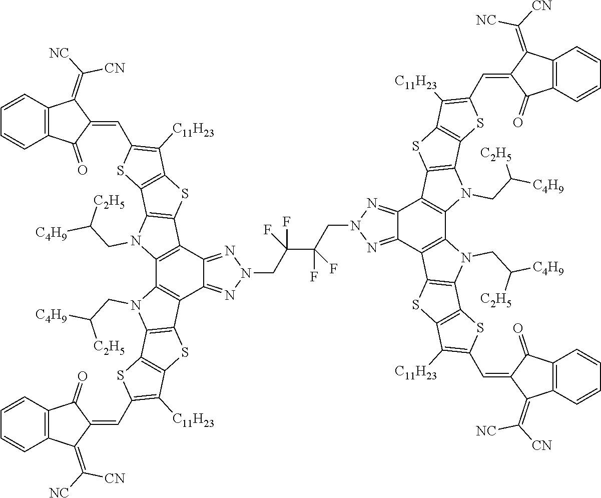 |
| N10: | 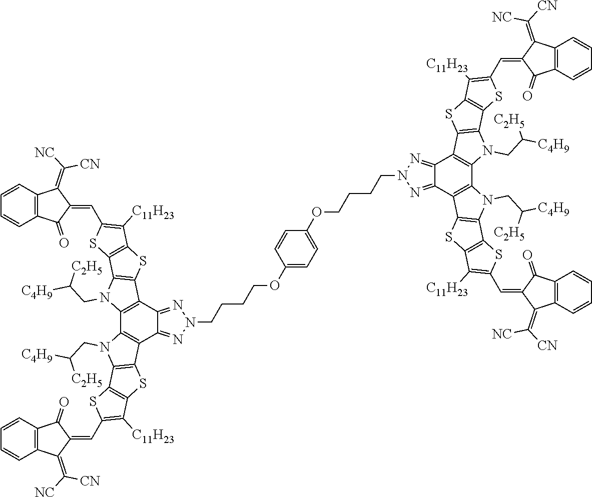 |
| N11: | 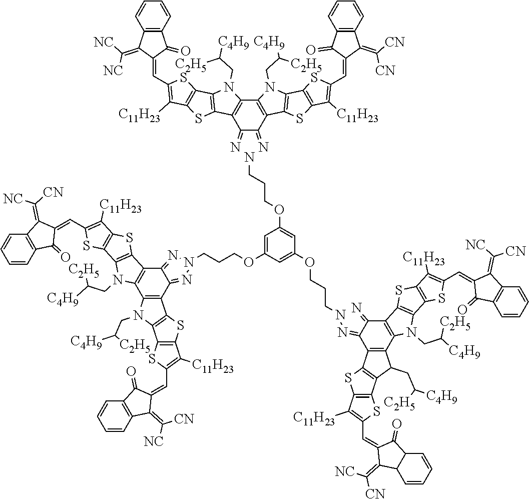 |
| N12: | 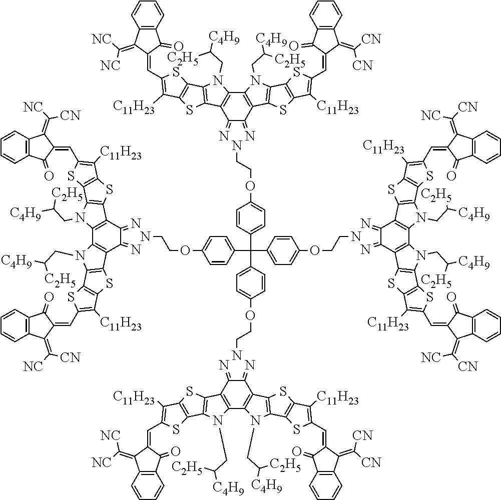 |

Furthermore, the organic semiconducting compound according to the present application is used as a charge transport, semiconducting, conductive, photoconductive, or light-emitting material in optical, electro-optical, electronic, electroluminescent, or photovoltaic components or devices. In these components or devices, the organic semiconducting compound according to the present application is usually used as a thin layer or film.

The organic semiconducting compound according to the present application is suitable as an electron acceptor or n-type semiconductor for organic photoelectric components, and is suitable for preparing blends of n-type and p-type semiconductors for use in the fields such as organic photodetector components. The term “n-type” or “n-type semiconductor” will be understood to refer to an extrinsic semiconductor in which the density of conducting electron exceeds the density of holes; and the term “p-type” or “p-type semiconductor” will be understood to mean extrinsic semiconductors in which the density of holes exceeds the density of conducting electrons. (Also refer to J. Thewlis, Concise Dictionary of Physics, Pergamon Press, Oxford, 1973)

When the organic semiconducting compound according to the present application is to be processed, one or more small molecule compounds and/or polymers with charge transport, semiconducting, conductive, photoconductive, hole-blocking and electron-blocking properties need to be added first and mixed to form a combination.

Moreover, the organic semiconducting compound according to the present application can be mixed with one or more organic solvents. The organic solvents are preferably aliphatic hydrocarbons, chlorinated hydrocarbons, aromatic hydrocarbons, ketones, ethers and mixtures thereof. More preferably, they are toluene, o-xylene, p-xylene, 1,3,5-trimethylbenzene or 1,2,4-trimethylbenzene, tetrahydrofuran, or 2-methyltetrahydrofuran.

The organic semiconducting compound according to the present application can be used to the patterned OSC layers in the devices as described herein. For modern microelectronics applications, it is generally desirable to produce small structures or patterns to reduce cost (more device/unit area) and power consumption. Patterning of thin layers containing the organic semiconducting compound according to the present application can be performed, for example, by photolithography, electron-beam etching techniques, or laser patterning.

For use as a thin layer in electronic or electro-optical devices, the first composition or the second composition formed by the organic semiconducting compound according to the present application can be deposited by any suitable method. Liquid coating of the device is better than the vacuum deposition technology. The second composition containing the organic semiconducting compound according to the present application can make the use of several liquid coating techniques feasible.

The preferred deposition techniques include, but not limited to, dip coating, spin coating, inkjet printing, nozzle printing, letterpress printing, screen printing, gravure printing, doctor blade coating, roller printing, reverse roller printing, lithography printing, dry lithography printing, quick drying printing, web printing, spray coating, curtain coating, brush coating, slot-dye coating, or pad printing.

The present application forms a composition, which includes an n-type organic semiconducting compound and a p-type organic semiconducting compound. The n-type organic semiconducting compound is the organic semiconducting compound of claim 1; the p-type organic semiconducting compound is a polymer.

In the composition according to the present application, the p-type organic semiconducting compound is selected from at least one of:

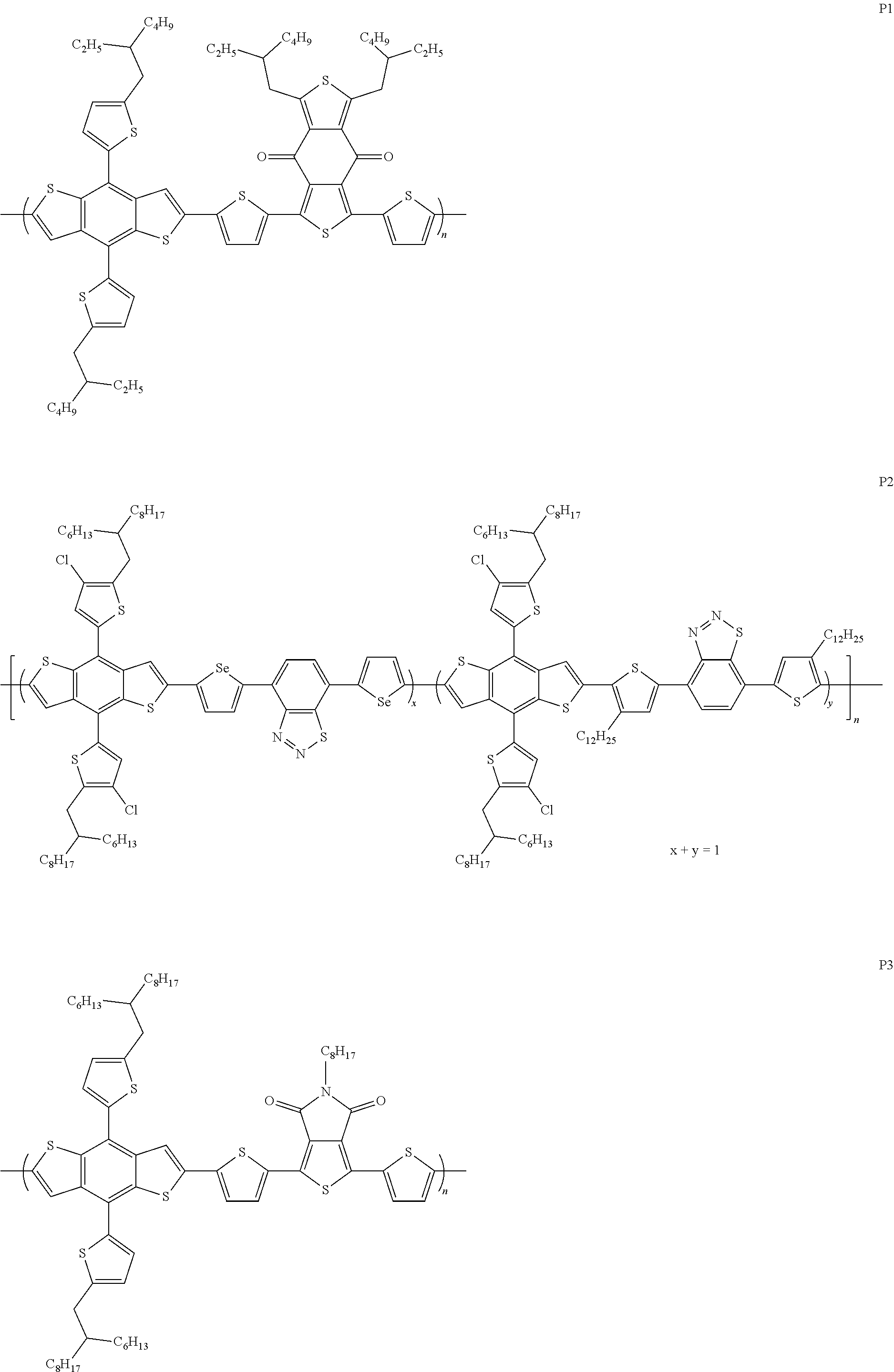

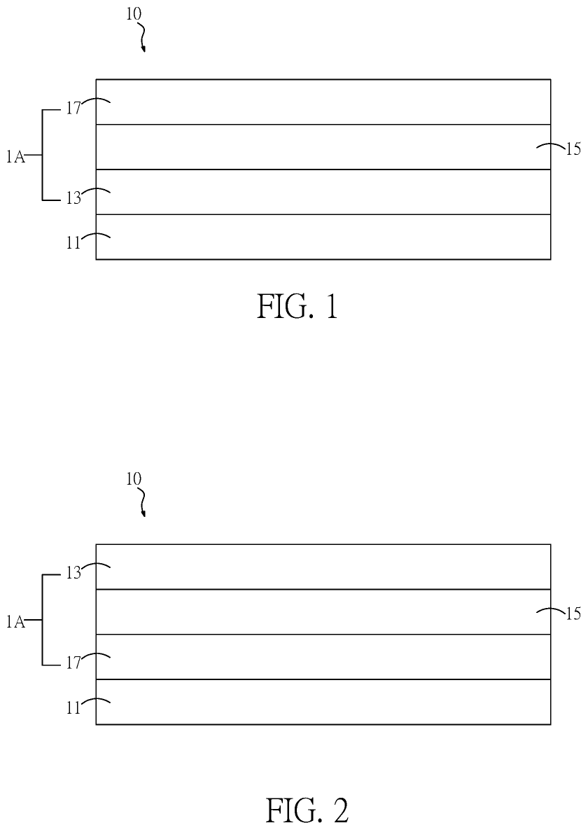

Moreover, please refer to FIG. 1, which shows a structural schematic diagram of the organic photoelectric component according to a first embodiment of the present application. As shown in the figure, the present application also provides an organic photoelectric component 10 containing the organic semiconducting compound, which comprises a substrate 11, an electrode module 1A, and an active layer 15.

According to the present embodiment, the electrode module 1A is disposed on the substrate 11 and includes a first electrode 13 and a second electrode 17. The active layer 15 is disposed between the first electrode 13 and the second electrode 17.

The material of the active layer 15 comprises at least one organic semiconducting compound of claim 1 or the composition of claim 7.

According to the present embodiment, the first electrode 13, the active layer 15, and the second electrode 17 are disposed bottom-up on the substrate 11 sequentially. In other words, the first electrode 13 is disposed on the substrate 11; the active layer 15 is disposed on the first electrode 13; and the second electrode 17 is disposed on the active layer 15.

Preferably, the substrate 11 adopts a glass substrate or a transparent flexible substrate that has mechanical strength, thermal strength, and transparency. The material of the transparent flexible substrate can be polyethylene, ethylene-vinyl acetate copolymer, ethylene-vinyl alcohol copolymer, polypropylene, polystyrene, polymethyl methacrylate, polyvinyl chloride, polyvinyl alcohol, poly Vinyl butyraldehyde, nylon, polyether ether ketone, polystyrene, polyether styrene, tetrafluoroethylene-perfluoroalkyl vinyl ether copolymer, polyfluoroethylene, tetrafluoroethylene-ethylene copolymer, tetrafluoroethylene-hexafluoropropylene copolymer, polychlorotrifluoroethylene, polyvinylidene fluoride, polyester, polycarbonate, polyurethane, polyimide, etc.

According to the present embodiment, one or more of the first electrode 13 and the second electrode 17 is transparent or translucent.

According to the present embodiment, the material of the first electrode 13 as described above is selected from at least one of halogen-doped or -undoped indium oxide, doped or undoped tin oxide, indium tin oxide or indium zinc oxide.

According to the present embodiment, the material of the second electrode 17 is selected from the compound or the composition of metal oxides, metals, conductive polymers, carbon-based conductors, and metallic compounds.

Please refer to FIG. 2, which shows a structural schematic diagram of the organic photoelectric component according to a second embodiment of the present application. As shown in the figure, the connection among the organic photoelectric component 10, the substrate 11, the electrode module 1A, and the active layer 15, and the materials of the substrate 11, the first electrode 13, the active layer 15, and the second electrode 17 are identical to the previous embodiment (the first embodiment). Hence, the details will not be repeated.

According to the present embodiment, the second electrode 17, the active layer 15, and the first electrode 13 are disposed bottom-up on the substrate 11 sequentially.

According to the present embodiment, the second electrode 17 is disposed on the substrate 11; the active layer 15 is disposed on the second electrode 17; and the first electrode 13 is disposed on the active layer 15.

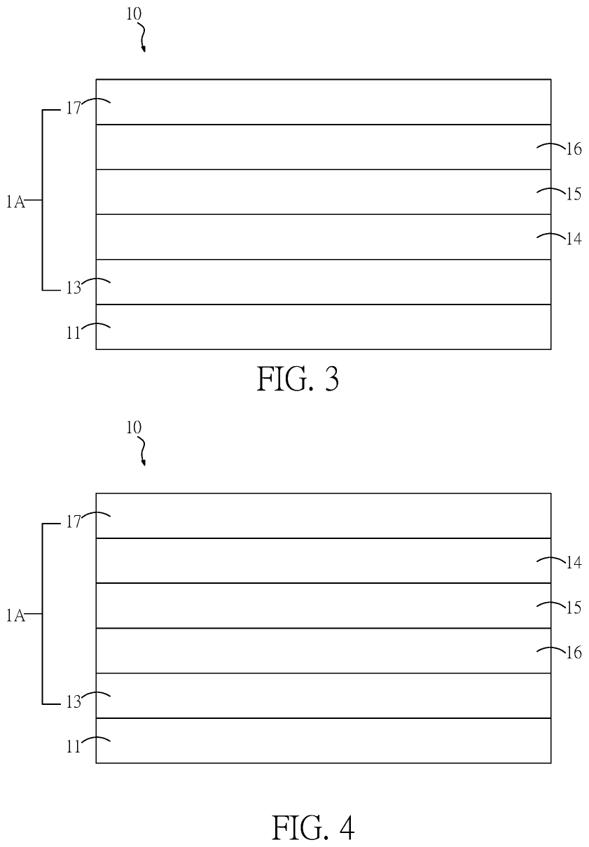

Please refer to FIG. 3, which shows a structural schematic diagram of the organic photoelectric component according to a third embodiment of the present application. As shown in the figure, the connection among the organic photoelectric component 10, the substrate 11, the electrode module 1A, and the active layer 15, and the materials of the substrate 11, the first electrode 13, the active layer 15, and the second electrode 17 are identical to the first embodiment. Hence, the details will not be repeated.

According to the present embodiment, the first electrode 13 is disposed on the substrate 11; the active layer 15 is disposed on the first electrode 13; and the second electrode 17 is disposed on the active layer 15.

According to the present embodiment, the organic photoelectric component further comprises a first carrier transport layer 14 and a second carrier transport layer 16. The first carrier transport layer 14 is disposed between the first electrode 13 and the active layer 15; the second carrier transport layer 16 is disposed between the active layer 15 and the second electrode 17.

According to the present embodiment, the material of the first carrier transport layer 14 is selected from the compound or the composition of conjugated polymer electrolyte, such as PEDOT:PSS, polymer acid, such as polyacrylate, conjugated polymer, such as polytriarylamine (PTAA), insulative polymer, such as Nafion, polyethylenimine, or polystyrene sulfonate, metal-oxide-doped polymer, metal oxide, and organic small-molecule compound, such as N,N′-diphenyl-N,N′-bis(1-naphthyl)(1,1′-biphenyl)-4,4′-diamine (NPB) or N,N′-diphenyl-N,N′-(3-methylphenyl)-1,1′-biphenyl-4,4′-diamine (TPD). The metal oxides include MoOₓ, NiOₓ, WOₓ, and SnOₓ. The material of the second carrier transport layer 16 is selected from the compound or the composition of conjugated polymer electrolyte, such as polyethyleneimine, conjugated polymer, such as poly[3-(6-trimethylammoniumhexyl)thiophene], poly(9,9)-bis(2-ethylhexyl-fluorene)-b-poly[3-(6-trimethylammonohexyl)thiophene], or Poly[(9,9-bis(3′-(N,N-dimethylamino)propyl)-2,7-fluorene)-alt-2,7-(9,9-dioctylfluorene)], organic small-molecule compound, such as tris(8-quinolyl)-aluminum(III)(Alq₃) or 4,7-diphenyl-1,10-phenanthroline, metal oxide, such as ZnOx, aluminum-doped ZnO (AZO), TiOₓ or its nanoparticles, salts, such as LiF, NaF, CsF, or Cs₂CO₃, and amine, such as primary, secondary, or tertiary amines.

Please refer to FIG. 4, which shows a structural schematic diagram of the organic photoelectric component according to a fourth embodiment of the present application. As shown in the figure, the connection among the organic photoelectric component 10, the substrate 11, the electrode module 1A, and the active layer 15, and the materials of the substrate 11, the first electrode 13, the active layer 15, and the second electrode 17 are identical to the first embodiment. The materials of the first carrier transport layer 14 and the second carrier transport layer 16 according to the present embodiment is identical to the previous embodiment (the third embodiment). Hence, the details will not be repeated.

According to the present embodiment, the first electrode 13 is disposed on the substrate 11; the active layer 15 is disposed on the first electrode 13; and the second electrode 17 is disposed on the active layer 15. The first carrier transport layer 14 is disposed between the second electrode 17 and the active layer 15; the second carrier transport layer 16 is disposed between the active layer 15 and the first electrode 13.

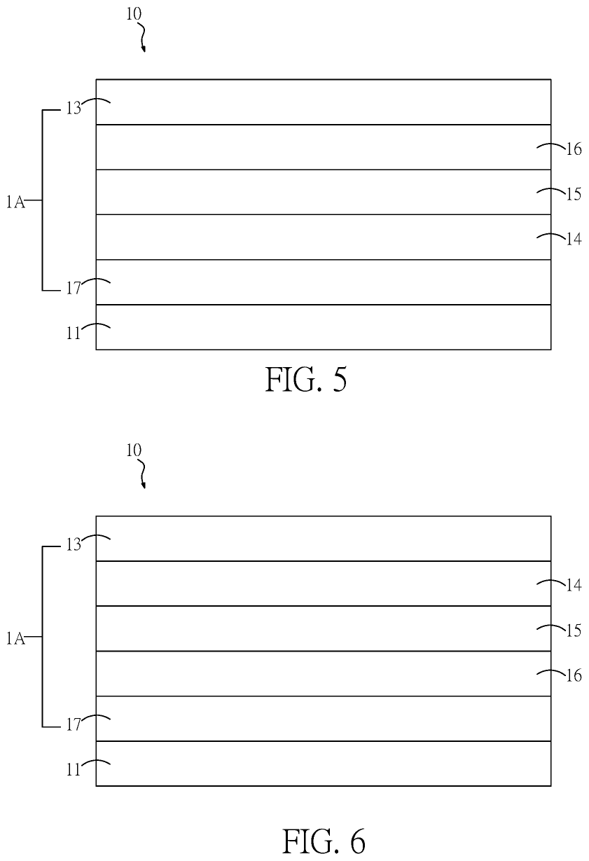

Please refer to FIG. 5, which shows a structural schematic diagram of the organic photoelectric component according to a fifth embodiment of the present application. As shown in the figure, the connection among the organic photoelectric component 10, the substrate 11, the electrode module 1A, and the active layer 15, and the materials of the substrate 11, the first electrode 13, the active layer 15, and the second electrode 17 are identical to the first embodiment. The materials of the first carrier transport layer 14 and the second carrier transport layer 16 according to the present embodiment is identical to the third embodiment. Hence, the details will not be repeated.

According to the present embodiment, the second electrode 17 is disposed on the substrate 11; the active layer 15 is disposed on the second electrode 17; and the first electrode 13 is disposed on the active layer 15. The first carrier transport layer 14 is disposed between the second electrode 17 and the active layer 15; the second carrier transport layer 16 is disposed between the active layer 15 and the first electrode 13.

Please refer to FIG. 6, which shows a structural schematic diagram of the organic photoelectric component according to a sixth embodiment of the present application. As shown in the figure, the connection among the organic photoelectric component 10, the substrate 11, the electrode module 1A, and the active layer 15, and the materials of the substrate 11, the first electrode 13, the active layer 15, and the second electrode 17 are identical to the first embodiment. The materials of the first carrier transport layer 14 and the second carrier transport layer 16 according to the present embodiment is identical to the third embodiment. Hence, the details will not be repeated.

According to the present embodiment, the second electrode 17 is disposed on the substrate 11; the active layer 15 is disposed on the second electrode 17; and the first electrode 13 is disposed on the active layer 15. The second carrier transport layer 16 is disposed between the second electrode 17 and the active layer 15; the first carrier transport layer 14 is disposed between the active layer 15 and the first electrode 13.

In order to illustrate the improvement in efficacy brought about by the application of the organic semiconducting compound according to the present application to organic photoelectric components, organic photoelectric components containing the organic semiconducting compound according to the present application are prepared and subjected to property testing. The test results are as follows:

### Embodiment 1: Thermal Stability Test of Compound Films

After heating and dissolving the compound in o-xylene (solid content: 20 mg/ml), the solution is spin-coated on the glass substrate with a spin coater in the atmosphere and baked at 100° ° C. for one minute. After cooling, measure the thin-film absorption and record the maximum absorption value. Second measurement: Bake the thin-film sample at 100° C. for 5 minutes in the atmosphere. After the sample cools down, measure the thin-film absorption and record the absorption value at the wavelength with the maximum absorption during the first measurement. Third measurement: Bake the sample at 160° C. for 5 minutes in the atmosphere. After the sample cools down, measure the thin-film absorption and record the absorption value at the wavelength with the maximum absorption during the first measurement. The difference between the second and third measurement values and the first measurement value divided by the first measurement value is the absorption change after baking. The results of absorption spectrum and electrochemical properties of samples are shown in Table 2.

The structures of the comparative compounds for testing are shown below:

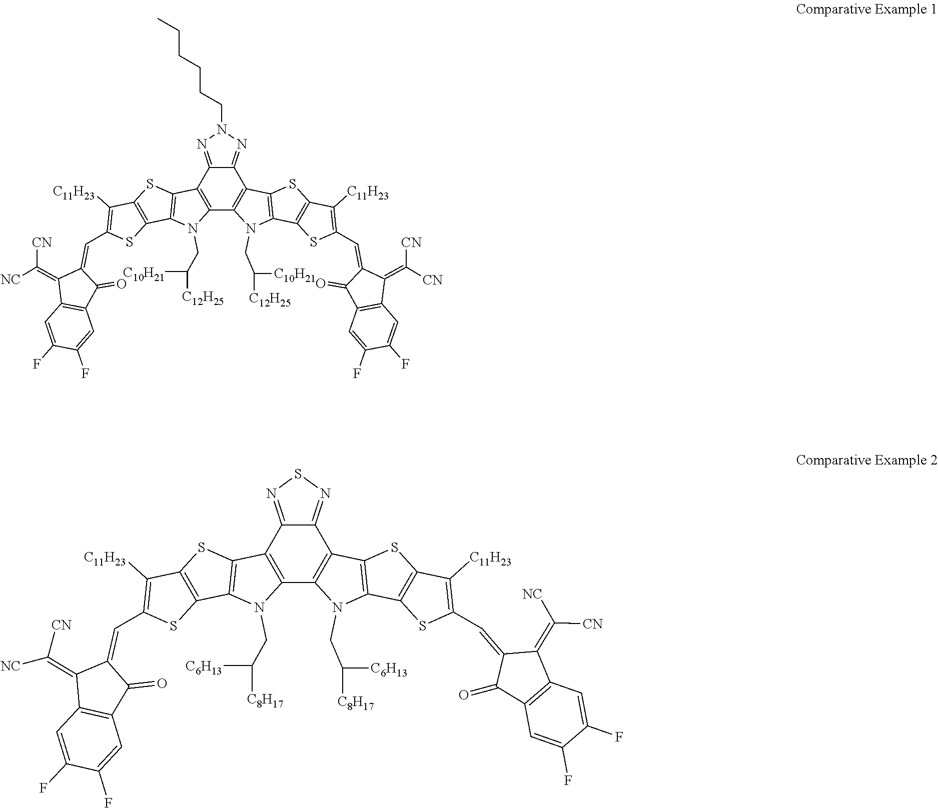

TABLE 2
Results of absorption spectrum and electrochemical properties of samples

| | λₛₒₗₙ^{max} | λ_{film}^{max} | λ_{film}^{onset} | ε | E_{g}^{opt} | HOMO | LUMO |
|---|---|---|---|---|---|---|---|
| Material | (nm) | (nm) | (nm) | (10⁵ cm⁻¹M⁻¹) | (eV) | (eV) | (eV) |
| N1 | 758 | 826 | 893 | 2.47 | 1.39 | −5.74 | −4.35 |
| N2 | 778 | 849 | 912 | 2.98 | 1.36 | −5.71 | −4.35 |
| N3 | 762 | 814 | 872 | 3.85 | 1.42 | −5.59 | −4.17 |
| Comparative | 760 | 862 | 906 | 2.32 | 1.37 | −5.67 | −4.30 |
| Example 1 | | | | | | | |
| Comparative | 743 | 835 | 919 | 1.86 | 1.35 | −5.72 | −4.37 |
| Example 2 | | | | | | | |

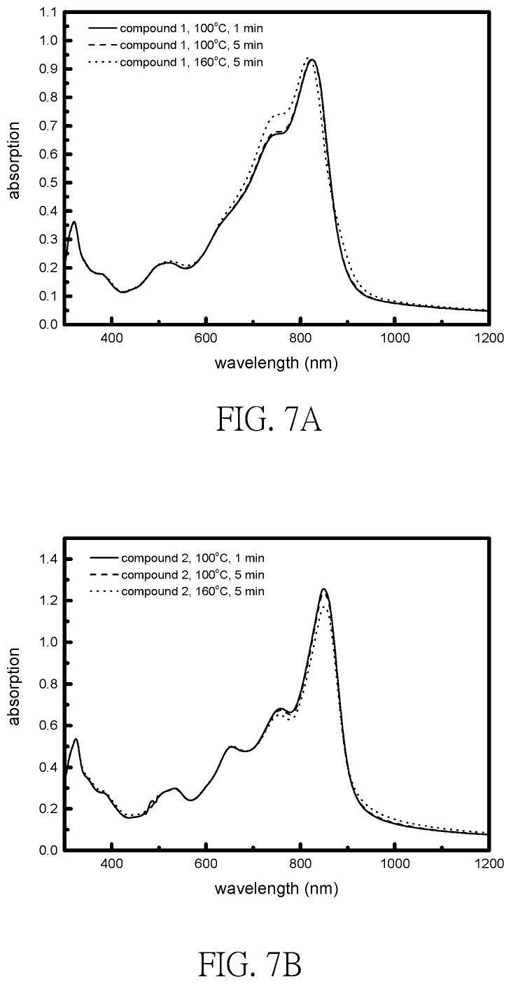

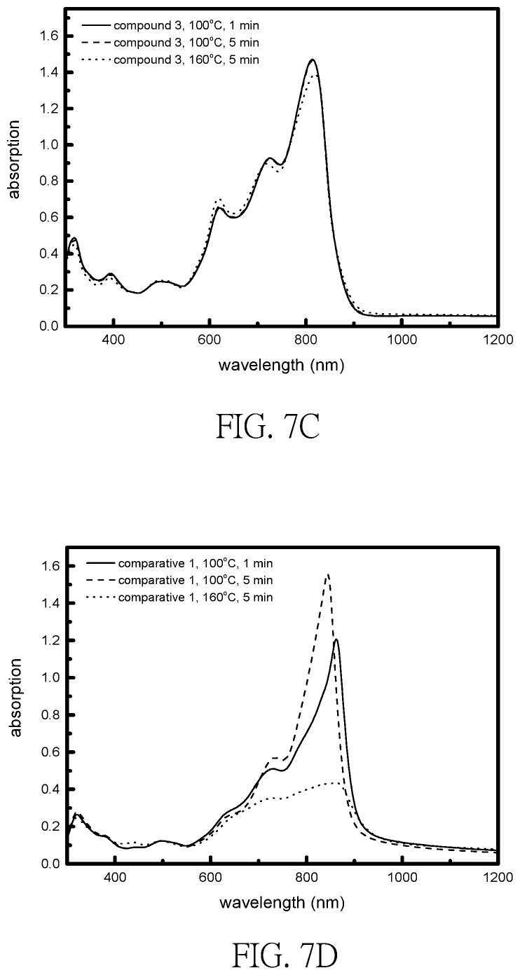

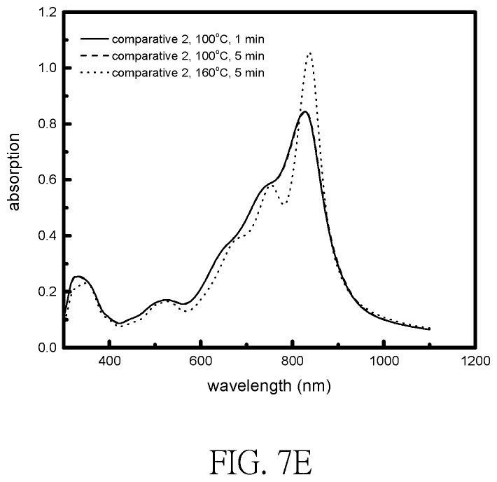

Please refer to FIG. 7A to FIG. 7E, which show the experimental results of absorption spectrum for the organic semiconducting compounds and the comparative compounds according to the present application. Referring to the experimental results in Table 2 above, it can be seen that the materials N1, N2, and N3 according to the present application have good performance in the absorption spectrum. Their maximum values of thin-film absorption fall at 758, 778, and 762 nm, respectively, and the initial absorption values fall at 893, 912, and 872 nm, respectively. It can be seen from the thin-film absorption spectrum that the three compounds still have good absorption properties at 600-900 nm after heating at 100° ° C. for 5 minutes, and their absorbance changes are about 0.2-1.4% after heating at 100° C. for 5 minutes. Furthermore, after heating at 160° C. for 5 minutes, the absorbance changes by about 0.6-6.9%. Compared with the compounds according to the present application, the maximum value of thin-film absorption of Comparative Example 1 has changed by 22.2% after heating at 100° C. for 5 minutes, and after heating at 160° C. for 5 minutes, Comparative Example 1 has changed by 64% while Comparative Example 2 shows a change of 18%. Both Comparative Example 1 and Comparative Example 2 show a change that is much greater than that of N1-N3. It is evident that the compounds according to the present application have better thermal stability than the compounds in the comparative examples.

### Embodiment 2: OPV Performance Tests

Pre-patterned ITO-coated glass with thin-film resistance is used as the substrate. Ultrasonic cleaning treatment is performed in neutral detergent, deionized water, acetone, and isopropyl alcohol sequentially for 15 minutes each. The washed substrate is further treated with UV-O₃ cleaner for 15 minutes. ZnO (diethyl zinc solution, 15 wt % in toluene, diluted with THF) are spin-coated on the ITO substrate at a spin rate of 5000 rpm for 30 seconds and then baked in air at 120° ° C. for 20 minutes. Prepare an active layer solution in o-xylene (the weight ratio of donor polymer: acceptor small molecule is 1:1.2˜1:1.5). The polymer concentration is 8˜10 mg/ml. In order to completely dissolve the polymer, the active layer solution should be stirred on a hot plate at 100° ° C. for at least 3 hours. The solution is then cooled to room temperature before coating, and the film thickness is controlled around 100 nm by spin rate. Afterwards, the film is annealed at 120° C. for 5 minutes and then transferred to the evaporator. Under vacuum plating of 3×10−6 Torr, deposit a thin layer of molybdenum trioxide (8 nm) as the anode intermediate layer. Use a solar simulator (xenon lamp with AM1.5G filter, 100 mW cm⁻²) in the air and measure the J-V characteristics of the device at room temperature. Here, a standard silicon diode with a KG5 filter is used as a reference cell to calibrate the light intensity so that the mismatched parts of the spectrum are consistent. Use the Keithley™ 2400 source meter to record the J-V characteristics. A typical cell has a device area of 4 mm², which is defined by a metal mask with openings aligned with the device area. PCE is the average of the measurement results of 4 effective points on each component. The test results are shown in Table 3.

TABLE 3
Performance tests on organic photovoltaic
cells with organic semiconducting compounds

| | | Open-circuit | Short-circuit | | | Average |
|---|---|---|---|---|---|---|
| | | voltage | current | Fill factor | Efficiency | Efficiency |
| Active layer | Ratio | (Volt) | (mA/cm²) | (%) | (%) | (%) |
| P2:Y6:N2 | 1:1:0.2 | 0.853 | 22.58 | 66.95 | 12.9 | 12.80 |
| P2:Y6:N3 | 1:1:0.2 | 0.862 | 22.46 | 66.61 | 12.9 | 12.64 |

It can be seen from the test results that when the compounds N2 and N3 according to the present application are used as active layer materials of organic photovoltaic cells, they can both achieve good power conversion efficiency of more than 12%.

### Embodiment 3: OPD Performance Tests

Pre-patterned ITO-coated glass with thin-film resistance is used as the substrate. Ultrasonic cleaning treatment is performed in neutral detergent, deionized water, acetone, and isopropyl alcohol sequentially for 15 minutes each. The washed substrate is further treated with UV-O3 cleaner for 15 minutes. AZO (Aluminum-doped zinc oxide nanoparticles) are spin-coated on the ITO substrate at a spin rate of 2000 rpm for 40 seconds and then baked in air at 120° ° C. for 5 minutes. Prepare an active layer solution in o-xylene (the weight ratio of donor polymer: acceptor small molecule is 1:0.8˜1:1.5). The polymer concentration is 12˜16 mg/ml. In order to completely dissolve the polymer, the active layer solution should be stirred on a hot plate at 100° C. for at least 3 hours and filtered with a PTFE filter membrane (pore size 0.45˜1.2 μm). Then the active layer solution is heated for 1 hour. The solution is then cooled to room temperature before coating, and the film thickness is controlled from 500 to 1000 nm by spin rate. Afterwards, the mixed film is annealed at 100° C. for 5 minutes and then transferred to the evaporator. Under vacuum plating of 3×10-6 Torr, deposit a thin layer of molybdenum trioxide (8 nm) as the anode intermediate layer. After baking the components at 160° C. for 30 minutes and 60 minutes, the dark current and external quantum efficiency of component samples are tested, respectively. Use the Keithley™ 2400 source meter to record the dark current (I_{D}, bias voltage −8V) in the absence of light, and then use a solar simulator (xenon lamp with AM1.5G filter, 100 mW cm⁻²) in the air and measure the photocurrent (Iₚₕ) characteristics of the device at room temperature. Here, a standard silicon diode with a KG5 filter is used as a reference cell to calibrate the light intensity so that the mismatched parts of the spectrum are consistent. The external quantum efficiency (EQE) uses an external quantum efficiency meter with a measurement range of 300˜1100 nm (bias voltage 0˜−8V). Silicon (300˜1100 nm) is used for light source calibration. The test results of the relative dark current and relative external quantum efficiency of each sample are shown in Table 4.

TABLE 4
Performance tests on organic photodetectors
with organic semiconducting compounds

| | | 160° C. | | | Relative external quantum | | |
|---|---|---|---|---|---|---|---|
| | | Baking | Relative dark current | | efficiency (850 nm) | | |
| Active layer | Ratio | time (min) | −4 V | −8 V | 0 V | −2 V | −4 V |
| P3: | 1:1 | 0 | 1 | 1 | 1 | 1 | 1 |
| Comparative | | 30 | 18.42 | 15.88 | 0.083 | 0.636 | 0.693 |
| example 2 | | 60 | 80.26 | 64.71 | 0.15 | 0.335 | 0.528 |
| P3: N2 | 1:1 | 0 | 1 | 1 | 1 | 1 | 1 |
| | | 30 | 2.95 | 3.25 | 0.603 | 0.717 | 0.774 |
| | | 60 | 5.00 | 6.14 | 0.556 | 0.668 | 0.724 |

It can be seen from Table 4 that after baking the organic photodetector device prepared in Comparative Example 2 at 160ºC for 30 minutes and 60 minutes, at −4V, the dark current increased by 18.42-80.26 times compared with before baking, and the external quantum efficiency is only 52.8% of the original value. For the organic photodetector device prepared from the compound N2 of the present application, after baking at 160° C. for 30 minutes and 60 minutes, at −4V, the dark current only increases by 2.95-5.0 times (wherein the dark current is 2.2×10⁻⁵ A/cm² under −4V bias), and maintains a certain external quantum efficiency. It can be seen that the compound of the present application has better thermal stability compared to the comparative example.

The above-mentioned examples show that when the organic semiconducting compound of the present application is used to prepare organic photoelectric components, it can not only be dissolved using non-halogen organic solvents (such as o-xylene), but also exhibits good processability and good solubility to solvents during the manufacturing process. It is also conducive to large-scale manufacturing using solution processing methods. Moreover, the excellent thermal stability of the compound can make organic photoelectric components have better heat resistance and maintain good device performance.

1. An organic semiconducting compound, comprising: 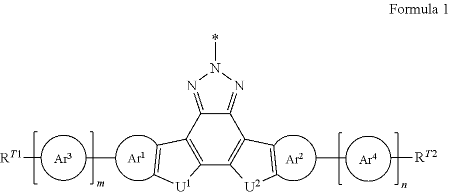
and a connecting unit, which is bonded to two or more units of Formula 1;
wherein
U¹, U² are NR¹, C═O, O, S, Se, or CR¹R²;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group;
Ar¹, Ar² are substituted or unsubstituted arylene or heteroarylene group with 5 to 20 ring atoms, which is a monocyclic, polycyclic, or condensed ring;
Ar³, Ar⁴ are independently selected from at least one of —CY¹═CY²—, —C≡C—, substituted monocyclic, polycyclic, or fused ring arylene with 5 to 20 ring atoms, or substituted monocyclic, polycyclic, or fused ring heteroarylene with 5 to 20 ring atoms;
Y¹, Y² are H, F, Cl, or CN;
R^{T1}, R^{T2} are electron-withdrawing group;
* is meaning a bonding position bonded to the connecting unit; and
m, n are integers from 0 to 5.

2. The organic semiconducting compound of claim 1, wherein said connecting unit is selected from at least one of: 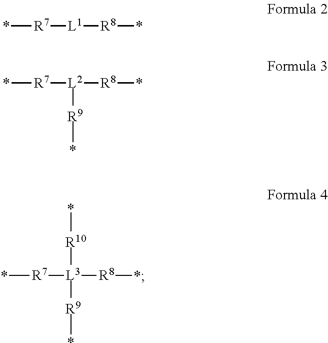
wherein
R⁷⁻¹⁰ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜ C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, and C3˜C30 alkyl group substituted by ketone group;
L¹ is selected from at least one of CR^{a}R^{b}, NR^{a}, O, SiR^{a}R^{b}, SO₂, (O═C)R^{a}R^{b} and A¹;
L² is selected from at least one of CR^{c}, N, SiR^{c}, P, P(O)₃, O═P(O)₃ and A²;
L³ is C, Si, A³;
* are bonding positions;
A¹-A³ are non-halogen-substituted aromatic ring groups with 5 to 20 ring atoms, monohalogen-substituted, or polyhalogen-substituted; and
R^{a}, R^{b}, R^{c} are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 5 to 20 ring atoms.

3. The organic semiconducting compound of claim 2, wherein said connecting unit is selected from at least one of: 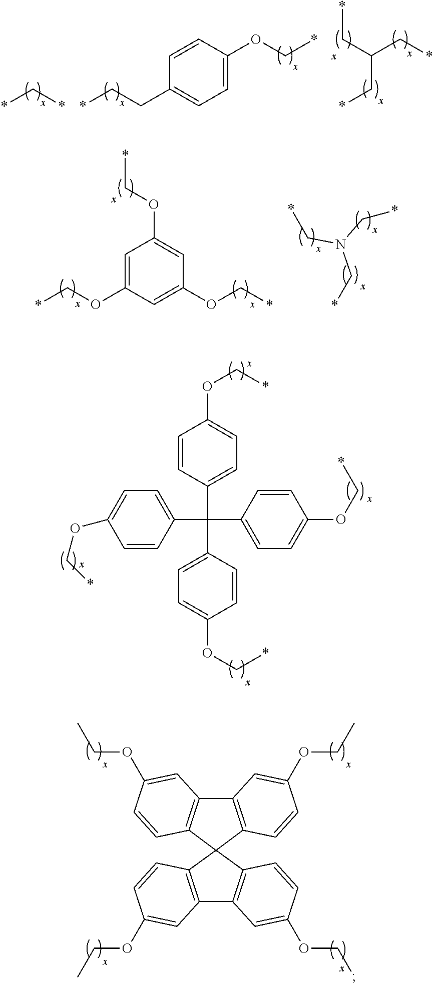
where x is an integer from 1 to 30, and * are bonding positions.

4. The organic semiconducting compound of claim 1, wherein Ar¹ is selected from at least one of: 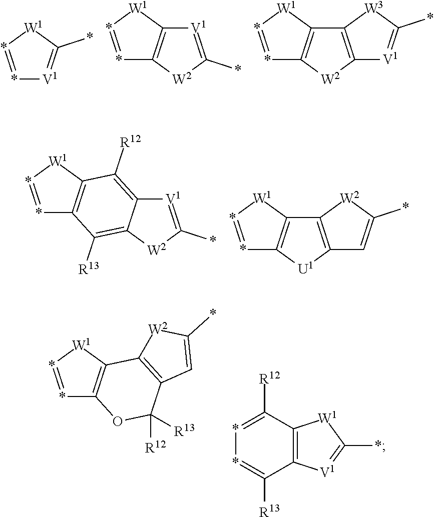
wherein
W¹⁻³ are O, S, or Se;
U¹ is NR¹, C═O, O, S, Se, or CR¹R²;
V¹ is N, CR¹;
* are bonding positions;
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
R¹²⁻¹³ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

5. The organic semiconducting compound of claim 4, wherein Ar¹ is selected from at least one of:

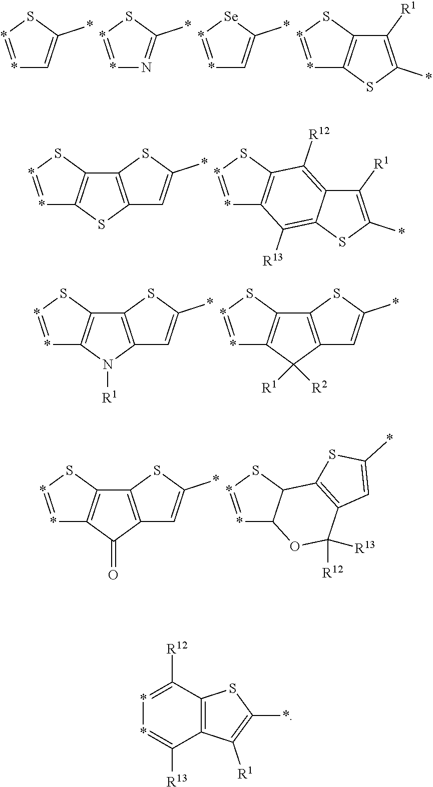

6. The organic semiconducting compound of claim 1, wherein Ar² is selected from at least one of: 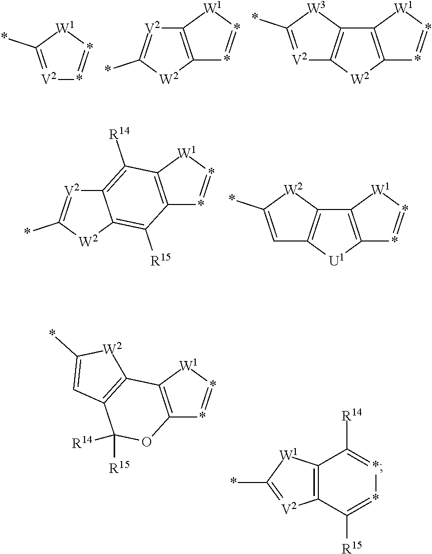
wherein
W¹⁻³ are O, S, or Se;
U¹ is NR¹, C═O, O, S, Se, or CR¹R²;
V² is N, CR¹;
* are meaning bonding positions; and
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; R¹⁴⁻¹⁵ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

7. The organic semiconducting compound of claim 6, wherein Ar² is selected from at least one of: 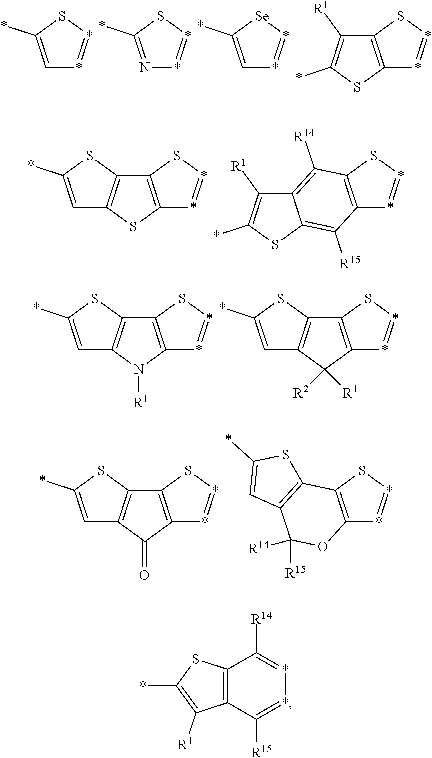
wherein
* are meaning bonding positions; and
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; R¹⁴⁻¹⁵ are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 6 to 18 ring atoms.

8. The organic semiconducting compound of claim 1, wherein Ar³⁻⁴ are selected from at least one of: 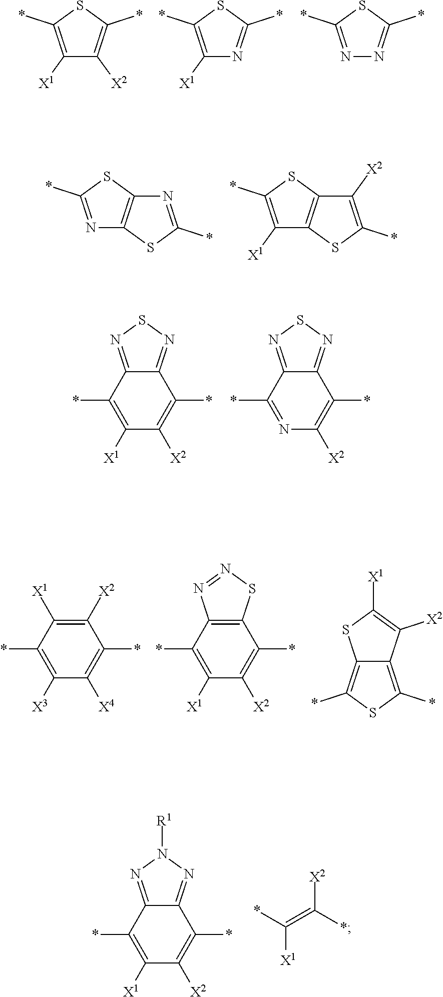
wherein
* are meaning bonding positions;
R¹ is independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group; and
X¹, X² are independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, and C3˜C30 alkyl group substituted by ketone group.

9. The organic semiconducting compound of claim 8, wherein Ar³⁻⁴ are selected from at least one of: 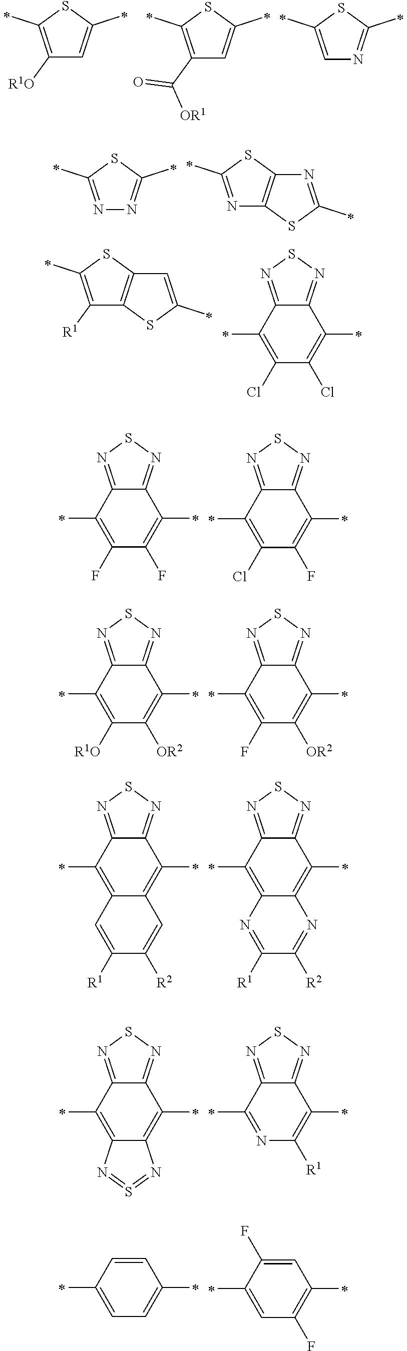 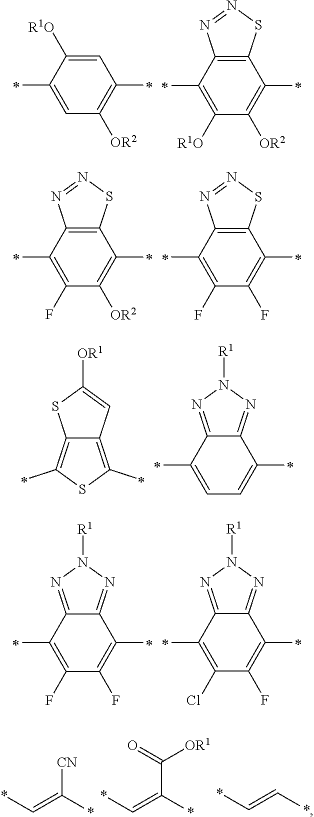
Wherein * are meaning bonding positions; and
R¹, R² are independently selected from at least one of hydrogen atom, C1˜C30 linear alkyl group, C3˜C30 branched-chain alkyl group, C1˜C30 silyl group, C2˜C30 ester group, C1˜C30 alkoxy group, C1˜C30 sulfoalkyl group, C1˜C30 haloalkyl group, C2˜C30 alkenyl group, and C2˜C30 alkynyl group.

10. The organic semiconducting compound of claim 1, wherein R^{T1} and R^{T2} are selected from at least one of: 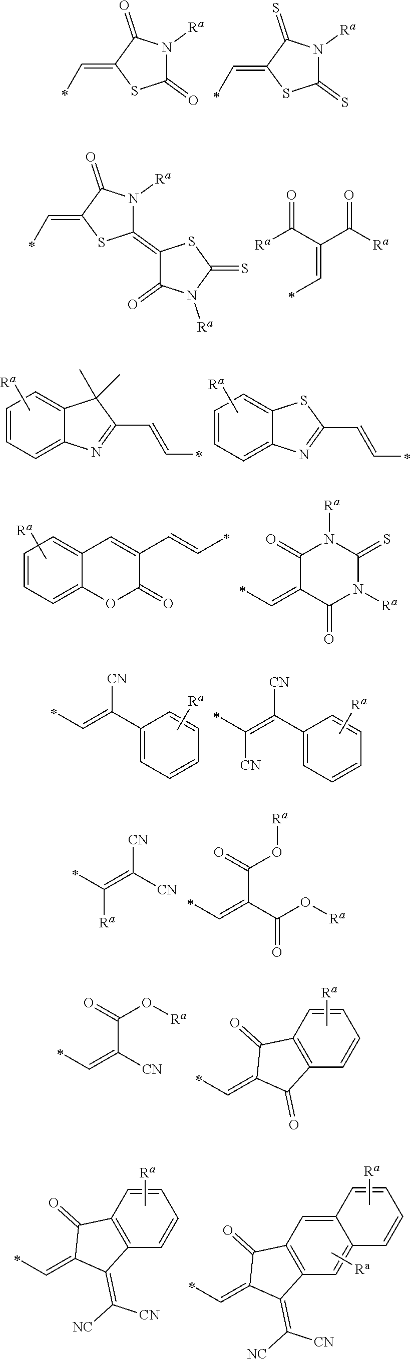 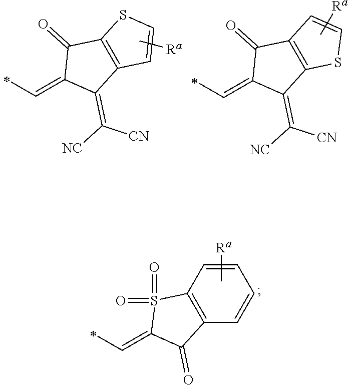
wherein * are meaning bonding positions; and
R^{a} is independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 5 to 20 ring atoms.

11. The organic semiconducting compound of claim 10, wherein R^{T1} and R^{T2} are selected from at least one of: 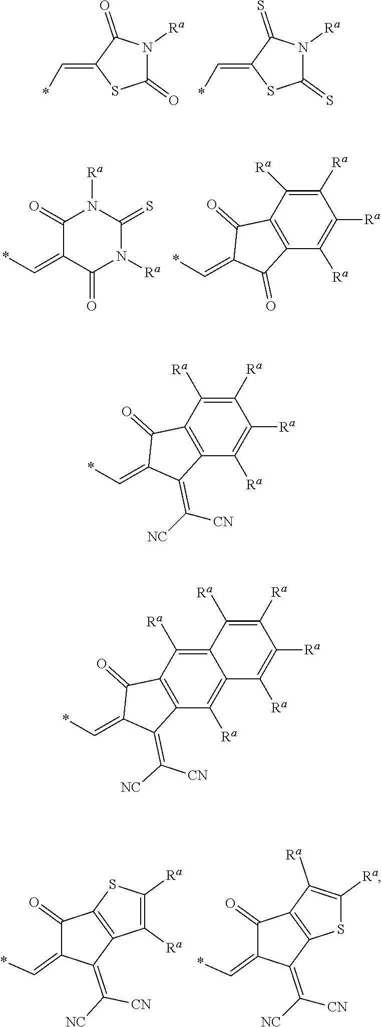
wherein * are meaning bonding positions; and
R^{a} is independently selected from at least one of hydrogen atom, halogen, cyano group, linear alkyl group of C1˜C30, branched-chain alkyl group of C3˜C30, silyl group of C1˜C30, ester group of C2˜C30, alkoxy group of C1˜C30, C1˜C30 thioalkyl, C1˜C30 haloalkyl, C2˜C30 alkenyl, C2˜C30 alkynyl, C2˜C30 cyano-substituted alkyl, C1˜C30 nitro-substituted alkyl group, C1˜C30 alkyl group substituted by hydroxyl group, C3˜C30 alkyl group substituted by ketone group, and substituted aryl or heteroaryl group of 5 to 20 ring atoms.

12. An organic photoelectric component, comprising:
a substrate;
an electrode module, disposed on said substrate, and including a first electrode and a second electrode; and
an active layer, disposed between said first electrode and said second electrode, said active layer comprising one or more n-type organic semiconducting compound selected from at least one of said organic semiconducting compound of claim 1;
wherein one or more of said first electrode and said second electrode is transparent or translucent.

13. The organic photoelectric component of claim 12, wherein said first electrode, said active layer, and said second electrode are disposed bottom-up on said substrate sequentially.

14. The organic photoelectric component of claim 12, wherein said second electrode, said active layer, and said first electrode are disposed bottom-up on said substrate sequentially.

15. The organic photoelectric component of claim 12, wherein said active layer further comprising one or more p-type organic semiconducting compound.

16. The organic photoelectric component of claim 12, further comprising:
a first carrier transport layer, disposed between said first electrode and said active layer; and
a second carrier transport layer, disposed between said second electrode and said active layer.

17. The organic photoelectric component of claim 12, further comprising:
a first carrier transport layer, disposed between said second electrode and said active layer; and
a second carrier transport layer, disposed between said first electrode and said active layer.

18. The organic photoelectric component of claim 16, wherein said first carrier transport layer is selected from at least one of SnO₂, ZnO, TiO₂, Cs₂CO₃, Nb₂O₅, PDMAEMA, and PFN-Br.

19. The organic photoelectric component of claim 17, wherein said first carrier transport layer is selected from at least one of SnO₂, ZnO, TiO₂, Cs₂CO₃, Nb₂O₅, PDMAEMA, and PFN-Br.

20. The organic photoelectric component of claim 16, wherein said second carrier transport layer is selected from at least one of PEDOT:PSS, MoO₃, NiO, V₂O₅, WO₃, CuSCN, spiro-MeOTAD, and PTAA.

21. The organic photoelectric component of claim 17, wherein said second carrier transport layer is selected from at least one of PEDOT:PSS, MoO₃, NiO, V₂O₅, WO₃, CuSCN, spiro-MeOTAD, and PTAA.
